(19) United States
(12) Patent Application Publication
(10) Pub. No.: US 2025/0109106 A1
(43) Pub. Date: Apr. 3, 2025

(54) Synthetic intermediates and improved processes for preparing ROCk inhibitors

(71) Applicant: Alcon Inc., Fribourg (CH)

(72) Inventors: Michael Scott McClure, Cary, NC (US); Daniel Francis Minkler, Raleigh, NC (US); Thomas Francis Nelson Haxell, Apex, NC (US)

(21) Appl. No.: 18/728,421

(22) PCT Filed: Nov. 2, 2022
(86) PCT No.: PCT/US2022/079176

§ 371 Date: Jul. 11, 2024

Related U.S. Application Data
(60) Provisional application No. 63/298,751, filed on Jan. 12, 2022.

(51) Int. Cl. C07D 217/16; C07C 68/08; C07C 211/63; C07C 211/64; C07C 229/38; C07D 295/023

(52) U.S. Cl. CPC C07D 217/16; C07C 68/08; C07C 211/63; C07C 211/64; C07C 229/38; C07D 295/023; C07C 2601/16

(57) ABSTRACT

Provided herein are methods of synthesizing netarsudil, and intermediates thereof, in high yield and at large commercial scale. A key intermediate in this synthesis is the preparation of piperidinium (S)-3-((tert-butoxycarbonyl)amino)-2-(4-(((2,4-dimethylbenzoyl)oxy)methyl)phenyl)propanoate, a salt that has been found to be easily purified by recrystallization.

### RELATED APPLICATIONS

This application is a national stage entry under 35 U.S.C. § 371 of international application no. PCT/US2022/079176, filed Nov. 2, 2022, which claims priority of U.S. 63/298,751, filed Jan. 12, 2022, the entire content of each of which is incorporated herein by reference.

### TECHNICAL FIELD

The present disclosure relates to a process for preparing compounds useful for treating kinase-related diseases and/or disorders. These include diseases and disorders of the eye, such as glaucoma, corneal disease, retinal disease, and ocular hypertension, diseases and conditions of the respiratory system, of the cardiovascular system, and for diseases characterized by abnormal growth, such as cancers.

### BACKGROUND

Rhopressa™ is a once-daily eye drop that inhibits, among other attributes, isoforms of Rho Kinase (ROCK), and is used for lowering intraocular pressure (IOP). It is thought that, by inhibiting these and other targets, Rhopressa™ reduces IOP via three separate mechanisms of action: (i) through ROCK inhibition, it increases fluid outflow through the trabecular meshwork, which accounts for approximately 80% of fluid drainage from the eye; (ii) it reduces episcleral venous pressure, which represents the pressure of the blood in the episcleral veins of the eye where eye fluid drains into the bloodstream; and (iii) through NET inhibition it may reduce the production of ocular fluid. Rhopressa™ has been approved by the FDA and EMA and is currently in phase 3 clinical trials in Japan for the treatment of glaucoma and ocular hypertension.

The active pharmaceutical ingredient of Rhopressa™ is (S)-4-(3-amino-1-(isoquinolin-6-ylamino)-1-oxopropan-2-yl)benzyl 2,4-dimethylbenzoate (netarsudil). Methods for its preparation are found in U.S. Pat. No. 8,394,826, which is hereby incorporated by reference in its entirety. Due to the clinical success of this compound, there exists a continuing need for improved processes to synthesize (S)-4-(3-amino-1-(isoquinolin-6-ylamino)-1-oxopropan-2-yl)benzyl 2,4-dimethylbenzoate (netarsudil) in an efficient, scaleable, and reproducible manner that will allow for the generation of large quantities of material with improved yields

### SUMMARY

Broadly, the instant disclosure extends to a method of synthesizing a compound of Formula (I):

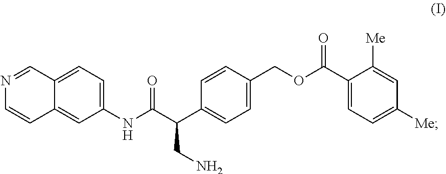 [SMILES: Cc1ccc(C(=O)OCc2ccc([C@@H](CN)C(=O)Nc3ccc4cnccc4c3)cc2)c(C)c1]

comprising:
(a) reacting a compound of Formula (II), wherein PG is a nitrogen protecting group,

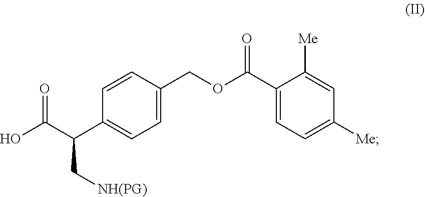

with 6-aminoisoquinoline to form a compound of Formula (III)

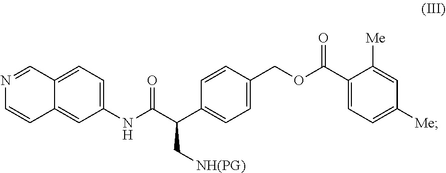

and
(b) removing the nitrogen protecting group to form the compound of Formula (I).

The instant disclosure further extends to a method of synthesizing a compound of Formula (I-a):

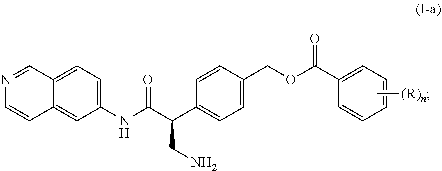

wherein each R is independently selected from the group consisting of C1-C4 alkyl, halogen, C1-C4 alkoxy and cyano; and n is an integer from 0 to 3; comprising:
(a) reacting a compound of Formula (II-a),

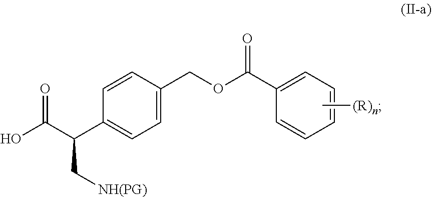

with 6-aminoisoquinoline to form a compound of Formula (III-a),

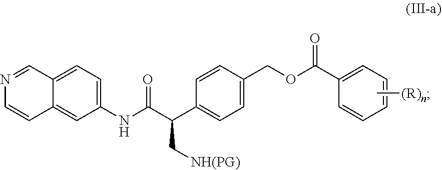

wherein each R is independently selected from the group consisting of C1-4 alkyl, halogen, C1-4 alkoxy and cyano; and n is an integer from 0 to 3; and
(b) removing the nitrogen protecting group to form the compound of Formula (I-a).

Also provided herein is a method of synthesizing a compound of Formula (XI):

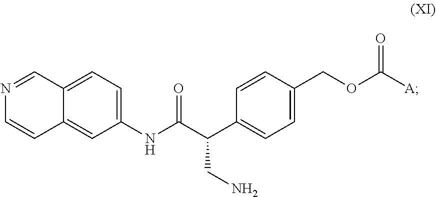

wherein A is cyclohexyl or phenyl, substituted with 0-3 substituents selected from the group consisting of alkyl, halogen, alkoxy, and cyano; comprising:
(a) reacting a compound of Formula (XII), wherein PG is a nitrogen protecting group,

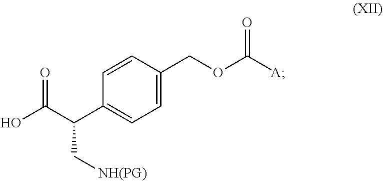

with 6-aminoisoquinoline to form a compound of Formula (XIII)

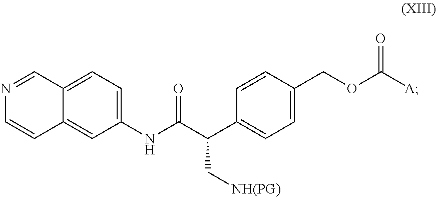

and
(b) removing the nitrogen protecting group to form the compound of formula (XI).

The instant disclosure further extends to a method of synthesizing a compound of formula (XI-a):

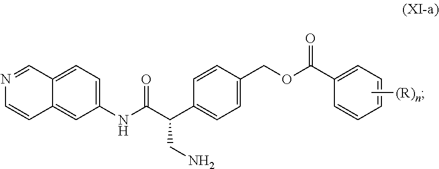

wherein each R is independently selected from the group consisting of C1-C4 alkyl, halogen, C1-C4 alkoxy and cyano; and n is an integer from 0 to 3;
   comprising:
(a) reacting a compound of formula (XII-a),

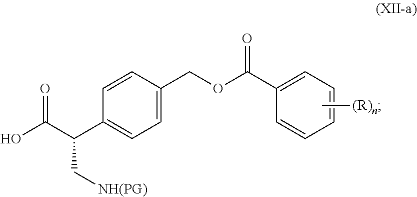

with 6-aminoisoquinoline to form a compound of formula (XIII-a),

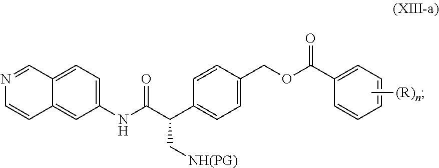

wherein each R is independently selected from the group consisting of C1-4 alkyl, halogen, C1-4 alkoxy and cyano; and n is an integer from 0 to 3; and
(b) removing the nitrogen protecting group to form the compound of formula (XI-a).

Also disclosed are pharmaceutical compositions comprising a compound of the instant disclosure as well as a pharmaceutically acceptable salt thereof, and methods of using a compound of the instant disclosure or a pharmaceutically acceptable salt thereof for treatment of kinase related diseases and/or disorders.

These and other aspects of the present disclosure will be better appreciated by reference to the following Detailed Description.

### DETAILED DESCRIPTION

Disclosed herein are processes for the synthesis of kinase inhibitors. The inhibitors can have Formula (I). Compounds of Formula (I) may by synthesized in a manner that efficiently generates large scale quantities of the compound of Formula (I). Compounds of Formula (I) can be used to treat or prevent kinase-related diseases and/or disorders. These include diseases and disorders of the eye, such as glaucoma, corneal damage, retinal inflammation and ocular hypertension, of the respiratory system, of the cardiovascular system, and for diseases characterized by abnormal growth, such as cancers.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl” as used herein, means a straight or branched, saturated hydrocarbon chain containing from 1 to 10 carbon atoms. The term “lower alkyl” or “C₁-C₆-alkyl” means a straight or branched chain hydrocarbon containing from 1 to 6 carbon atoms. The term “C₃-C₇ branched alkyl” means a branched chain hydrocarbon containing from 3 to 7 carbon atoms. The term “C₁-C₄-alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. An alkyl group can be substituted or unsubstituted.

The term “alkylene”, as used herein, refers to a divalent group derived from a straight or branched chain hydrocarbon of 1 to 10 carbon atoms, for example, of 2 to 5 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—. An alkylene group can be substituted or unsubstituted.

The term “alkenyl” as used herein, means a straight or branched, unsaturated hydrocarbon chain containing at least one carbon-carbon double bond and from 1 to 10 carbon atoms. The term “lower alkenyl” or “C₂-C₆-alkenyl” means a straight or branched chain hydrocarbon containing at least one carbon-carbon double bond and from 1 to 6 carbon atoms. An alkenyl group can be substituted or unsubstituted.

The term “alkoxyalkyl” as used herein, refers to an alkoxy group, as defined herein, appended to the parent molecular moiety through an alkyl group, as defined herein.

The term “alkynyl” as used herein, means a straight or branched, unsaturated hydrocarbon chain containing at least one carbon-carbon triple bond and from 1 to 10 carbon atoms. The term “lower alkynyl” or “C₂-C₆-alkynyl” means a straight or branched chain hydrocarbon containing at least one carbon-carbon triple bond and from 1 to 6 carbon atoms. An alkynyl group can be substituted or unsubstituted.

The term “aryl” as used herein, refers to a phenyl group, or a bicyclic fused ring system. Bicyclic fused ring systems are exemplified by a phenyl group appended to the parent molecular moiety and fused to a cycloalkyl group, as defined herein, a phenyl group, a heteroaryl group, as defined herein, or a heterocycle, as defined herein. Representative examples of aryl include, but are not limited to, indolyl, naphthyl, phenyl, quinolinyl and tetrahydroquinolinyl. An aryl group can be substituted or unsubstituted.

The term “cycloalkyl” as used herein, refers to a carbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl and cyclodecyl. A cycloalkyl group can be substituted or unsubstituted.

The term “cycloalkenyl” as used herein, refers to a carbocyclic ring system containing three to ten carbon atoms, zero heteroatoms and at least one double bond. A cycloalkenyl group can be substituted or unsubstituted.

The term “fluoroalkyl” as used herein, refers to at least one fluorine atom appended to the parent molecular moiety through an alkyl group, as defined herein. Representative examples of fluoroalkyl include, but are not limited to, trifluoromethyl.

The term “fluoroalkoxy” as used herein, refers to at least one fluorine atom appended to the parent molecular moiety through an alkoxy group, as defined herein.

The term “alkoxyfluoroalkyl” as used herein, refers to an alkoxy group, as defined herein, appended to the parent molecular moiety through a fluoroalkyl group, as defined herein.

The term “halogen” or “halo” as used herein, means Cl, Br, I, or F.

The term “haloalkyl” as used herein, refers to at least one halogen atom appended to the parent molecular moiety through an alkyl group, as defined herein.

The term “heteroalkyl” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides. A heteroalkyl group can be substituted or unsubstituted.

The term “heteroaryl” as used herein, refers to an aromatic monocyclic ring or an aromatic bicyclic ring system. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Representative examples of heteroaryl include, but are not limited to, indolyl, pyridinyl (including pyridin-2-yl, pyridin-3-yl, pyridin-4-yl), pyrimidinyl, thiazolyl, and quinolinyl. A heteroaryl group can be substituted or unsubstituted.

The term “heterocycle” or “heterocyclic” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. A heterocylic group can be substituted or unsubstituted.

The term “heteroarylalkyl” as used herein, refers to a heteroaryl group, as defined herein, appended to the parent molecular moiety through an alkyl group, as defined herein.

The term “hydroxyalkyl” as used herein, refers to a hydroxy group appended to the parent molecular moiety through an alkyl group, as defined herein

The term “arylalkyl” as used herein, refers to an aryl group, as defined herein, appended to the parent molecular moiety through an alkyl group, as defined herein.

In some instances, the number of carbon atoms in a hydrocarbyl substituent (e.g., alkyl or cycloalkyl) is indicated by the prefix “Cr-Cy-”, wherein x is the minimum and y is the maximum number of carbon atoms in the substituent. Thus, for example, “C₁-C₃-alkyl” refers to an alkyl substituent containing from 1 to 3 carbon atoms.

The term “substituents” refers to a group “substituted” on group at any atom of that group. Any atom can be substituted.

The term “substituted” refers to a group that may be further substituted with one or more non-hydrogen substituent groups. Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Processes

### Compounds of Formula (I)

In one aspect, disclosed is a process for the synthesis of the compound of Formula (I):

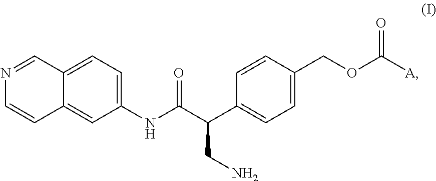

or a pharmaceutically acceptable salt; wherein A is cyclohexyl or phenyl, substituted with 0-3 substituents selected from the group consisting of alkyl, halogen, alkoxy, and cyano.

The process includes reacting 6-aminoisoquinoline with the compound of Formula (II), wherein PG is a protecting group for the nitrogen, to form the compound of Formula (III). The compound of Formula (III) can be transformed to the compound of Formula (I) by removal of the nitrogen protecting group. The nitrogen protecting group, PG, may be any suitable nitrogen protecting group known in the art. In certain embodiments, PG is selected from the group consisting of tert-butyloxycarbonyl (Boc), carbobenzyloxy (CBZ), 9-Fluorenylmethyloxycarbonyl (Fmoc), and para-methoxybenzyl carbonyl (Moz).

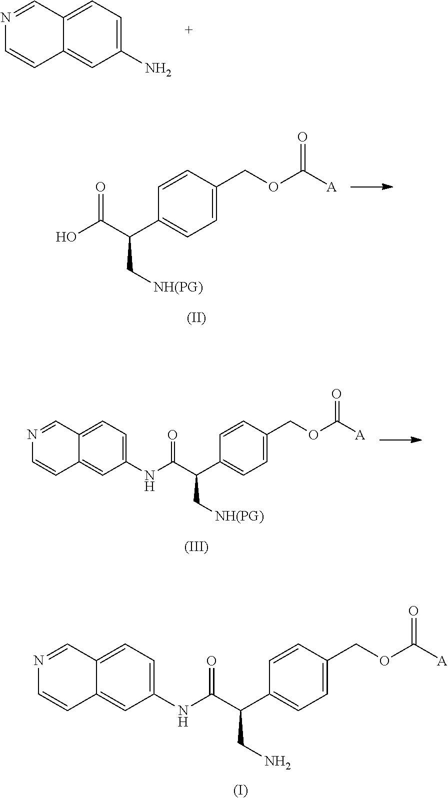

The process further includes the synthesis of the compound of Formula (II). Aminoalkylation of the compound of formula (IV), wherein T is a chiral auxiliary, can provide the compound of Formula (V), which can be converted to the compound of Formula (II) upon removal of the chiral auxiliary.

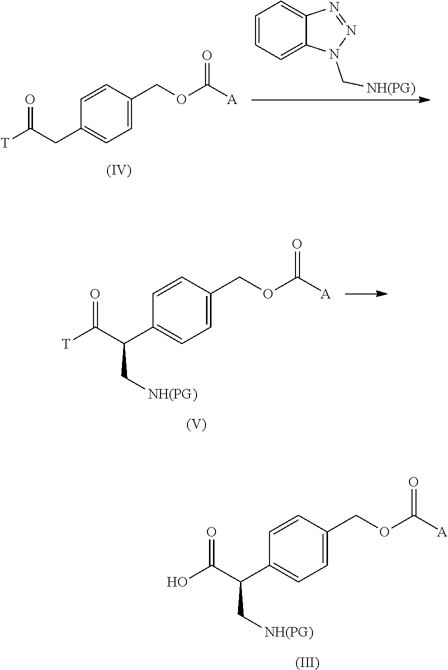

The compound of form Formula Ia (VII) can be prepared by reaction of methyl 2-(4-(hydroxymethyl)phenyl)acetate with the compound of Formula (VI). The compound of Formula (VII) can be converted to the compound of Formula (VIII), wherein R¹ is halogen, OR^{a}, OC(O)R^{b}, SR^{a}, or SC(O)R^{b}; wherein R^{a} is H, alkyl, or aryl, and R^{b} is alkyl or aryl. The compound of Formula (IV) can be obtained in turn from the compound of Formula (VIII), wherein T is a chiral auxiliary.

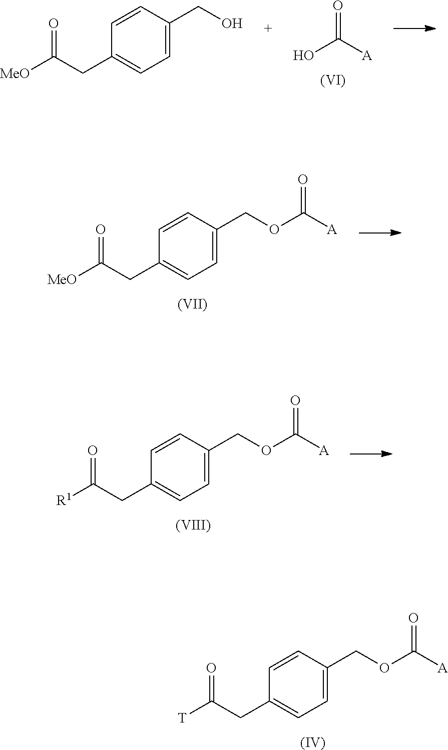

### Compounds of Formula (I-a)

In an embodiment, the process for the synthesis of the compound of Formula (I) is the process for the synthesis of the compound of Formula (I-a):

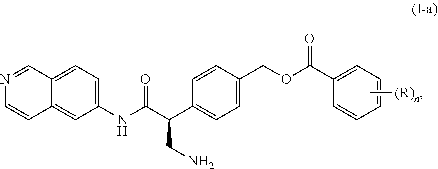

or a pharmaceutically acceptable salt; wherein each R is independently selected from the group consisting of C₁-C₄ alkyl, halogen, C₁-C₄ alkoxy and cyano; and n is an integer from 0 to 3. In some embodiments, the C₁-C₄ alkyl may be a C₁-C₄ fluoroalkyl.

The process includes reacting 6-aminoisoquinoline with the compound of Formula (II-a), wherein PG is a protecting group for the nitrogen, to form the compound of Formula (III-a). The compound of Formula (III-a) can be transformed to the compound of Formula (I-a) by removal of the nitrogen protecting group. The nitrogen protecting group, PG, may be any suitable nitrogen protecting group known in the art. In certain embodiments, PG is selected from the group consisting of tert-butyloxycarbonyl (Boc), carbobenzyloxy (CBZ), 9-Fluorenylmethyloxycarbonyl (Fmoc), and para-methoxybenzyl carbonyl (Moz).

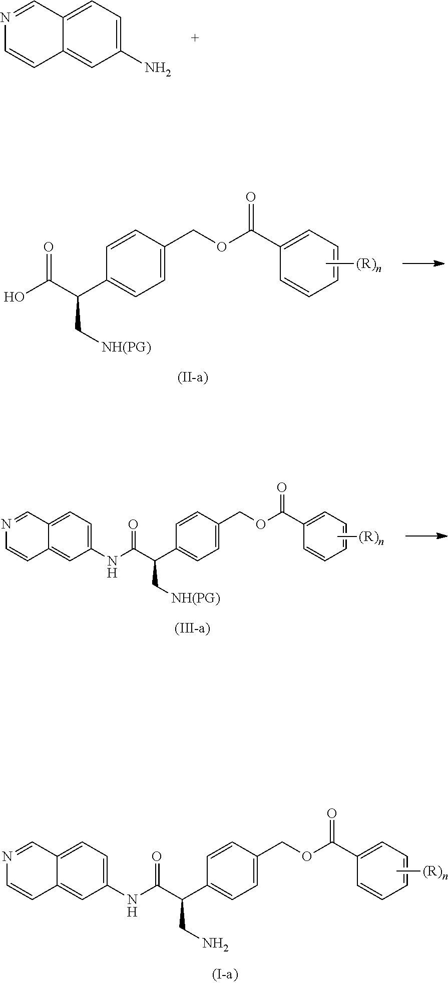

The process further includes the synthesis of the compound of Formula (II-a). Aminoalkylation of the compound of Formula (IV-a), wherein T is a chiral auxiliary, can provide the compound of Formula (V-a), which can be converted to the compound of Formula (II-a) upon removal of the chiral auxiliary.

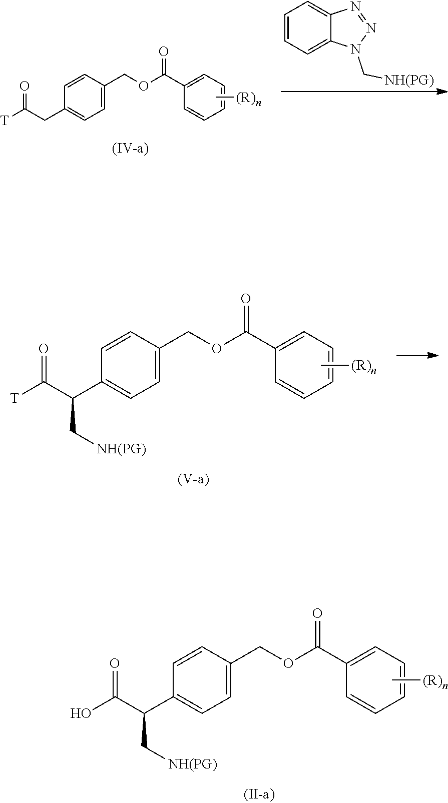

The compound of formula (VII-a) can be prepared by reaction of methyl 2-(4-(hydroxymethyl)phenyl)acetate with the compound of Formula (VI-a). The compound of Formula (VII-a) can be converted to the compound of Formula (VIII-a), wherein R¹ is halogen, OR^{a}, OC(O)R^{b}, SR^{a}, or SC(O)R^{b}; wherein R^{a} is H, alkyl or aryl, and R^{b} is alkyl or aryl. The compound of formula (IV-a) can be obtained in turn from the compound of Formula (VIII-a), wherein T is a chiral auxiliary.

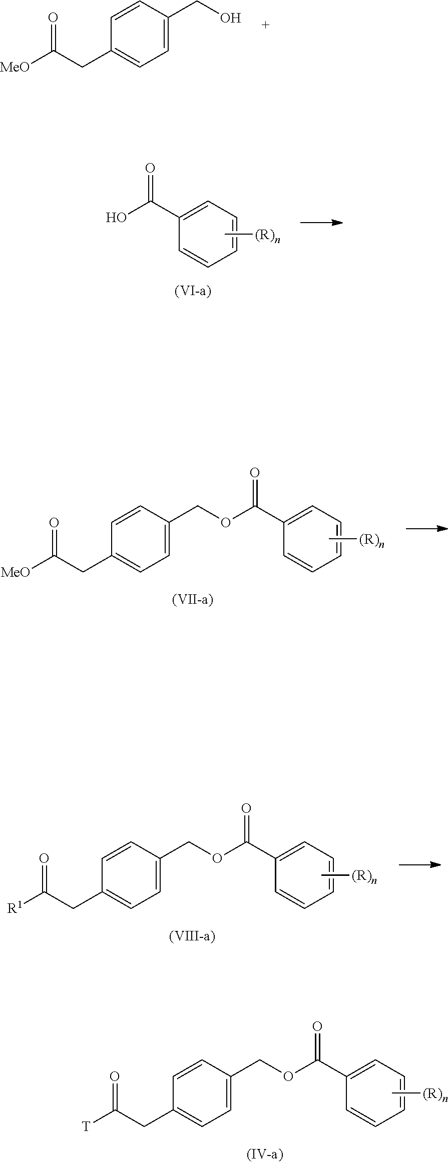

In certain embodiments, T may be the compound of formula (IX),

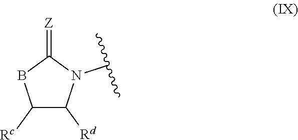

wherein Z is S or O; B is S or O; R^{c} is hydrogen, cycloalkyl, C₃-C₇ branched alkyl or aryl; R^{d} is C₁-C₄ alkyl, C₃-C₇ branched alkyl; arylalkyl or aryl; and R^{c} is C₁-C₄ alkyl or aryl.

Specifically, T may be the compound of Formula (IX-a)

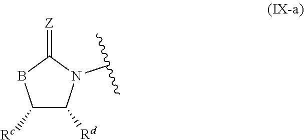

wherein Z is S or O; B is S or O; R^{c} is hydrogen or aryl; R^{d} is C₁-C₄ alkyl, arylalkyl or aryl; and R^{c} is C₁-C₄ alkyl or aryl.

More specifically, T may be selected from the group consisting of

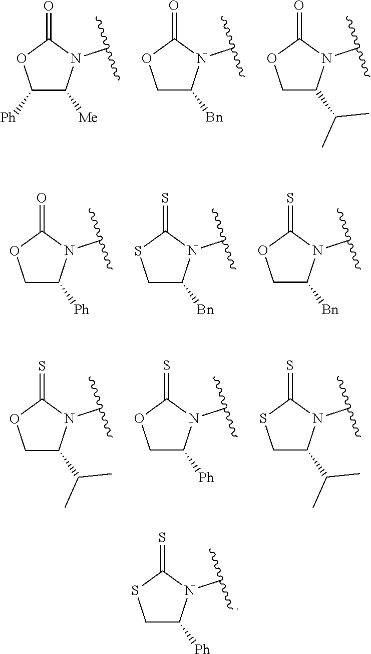

In a specific embodiment, T is

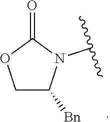 [SMILES: *N1C(=O)OC[C@H]1Cc1ccccc1]

### Compound (1)

In an embodiment, the disclosed process for the synthesis of the compound of Formula (I) is the process for the synthesis of compound (1):

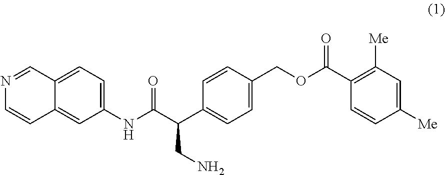 [SMILES: Cc1ccc(C(=O)OCc2ccc([C@@H](CN)C(=O)Nc3ccc4cnccc4c3)cc2)c(C)c1]

or a pharmaceutically acceptable salt.

The process includes reacting 6-aminoisoquinoline with compound (2) to form compound (3). Compound (3) can be transformed to compound (1) by removal of the Boc protecting group.

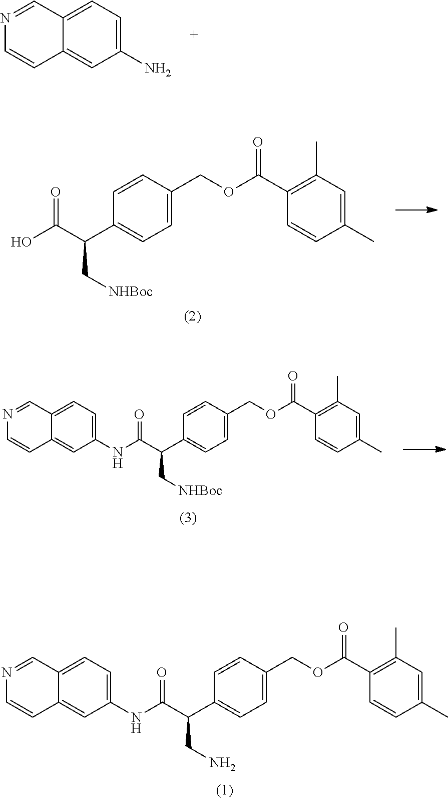

The process further includes the synthesis of compound (2). Aminoalkylation of compound (4) can provide compound (5), which can be converted to compound (2) upon removal of the chiral auxiliary.

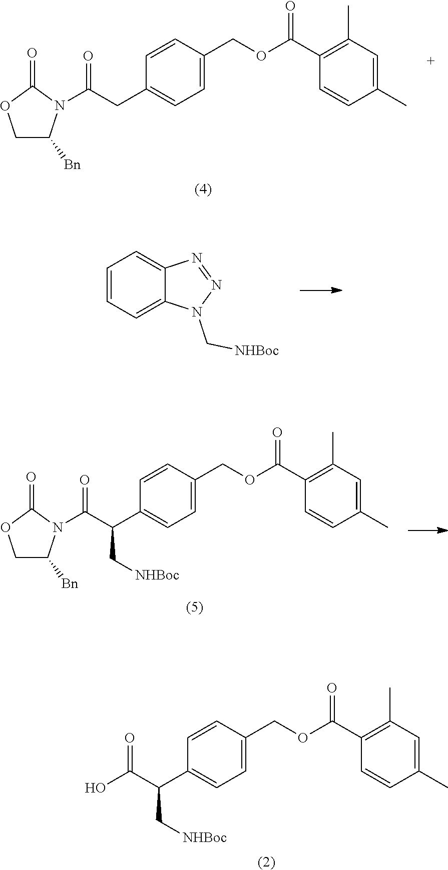

Compound (4) can be prepared by reaction of methyl 2-(4-(hydroxymethyl)phenyl) acetate with compound (6). Compound (7) can be converted to compound (8). Compound (4) can be obtained in turn from compound (8).

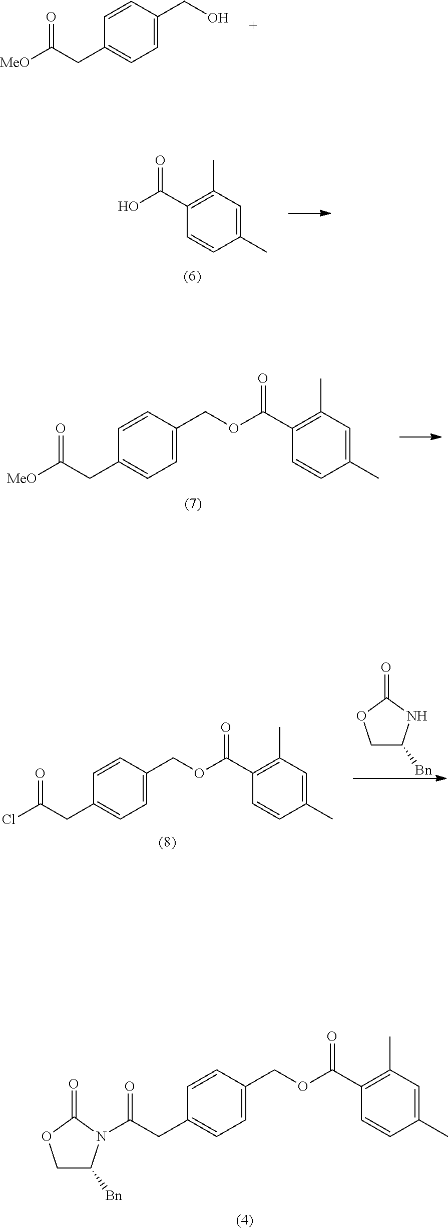

In a specific embodiment of the process for the synthesis of the compound of Formula (I) (e.g. compound (1)), the process includes the coupling of methyl 2-(4-(hydroxymethyl)phenyl) acetate with 2,4-dimethylbenzoic acid (6) in the presence of EDC and DMAP to form compound (7). The methyl ester of compound (7) can be selectively hydrolyzed with a suitable base (e.g. metal hydroxide such as lithium hydroxide, sodium hydroxide or potassium hydroxide) in a suitable solvent to yield compound (9). Suitably, the hydrolysis conditions include lithium hydroxide as base and a mixture of THF and water as solvent. These conditions are advantageous because they help limit the amount of hydrolysis of the benzylic ester.

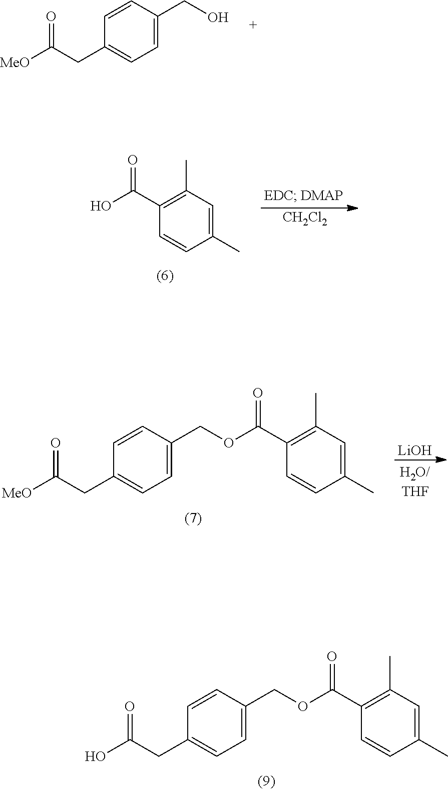

Compound (9) can be transformed to acid chloride (8) by treatment with a chlorinating agent. The chlorinating agent may be oxalyl chloride or thionyl chloride. The solvent may be a chlorinated solvent such as methylene chloride, dichloroethane or chloroform, or it may be a non-chlorinated solvent such as THF, diethyl ether, dioxane or acetonitrile. The chlorinating agent and solvent may be thionyl chloride. Suitably, the chlorinating agent is oxalyl chloride and the solvent is methylene chloride or a tetrahydofuran/dimethylformamide solvent mixture.

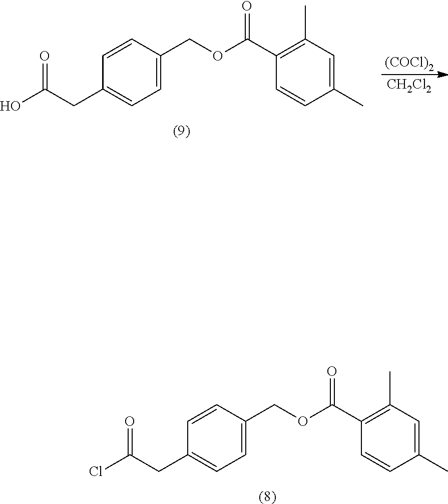

Addition of a base to (R)-4-benzyloxazolidin-2-one can be followed by reaction with compound (8) at a temperature of −90° C. to −50° C. to provide compound (4). The base used for addition to (R)-4-benzyloxazolidin-2-one may be NaH, LiH, KH, nBuLi, NaHMDS, LDA, triethylamine, ethyl diisopropylamine, methyl magnesium bromide, sodium carbonate, cesium carbonate, sec-BuLi, LiHMDS, potassium t-butoxide, sodium isopropoxide or KHMDS. The solvent may be THF, toluene, diethyl ether, acetonitrile, methyl t-butyl ether or a combination thereof. Suitably, the base used for addition to (R)-4-benzyloxazolidin-2-one is n-BuLi and the solvent is THF.

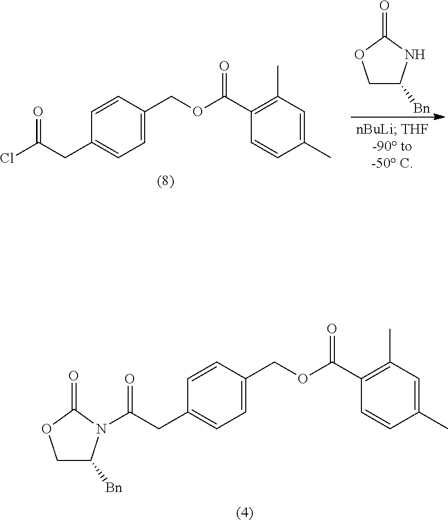

Compound (4) can be treated with a base followed by addition of N-Boc-1-aminomethylbenzotriazole at a temperature of −50° C. to −20° C. to provide compound (5) in a diastereoselective fashion. The base used for treatment of compound (4) may be LiHMDS, LDA, or NaHMDS. The solvent may be THF, toluene, diethyl ether, acetonitrile, methyl t-butyl ether or a combination thereof. Suitably, the base used for treatment of compound (4) is LiHMDS and the solvent is THF. In some embodiments, a Lewis acid may be added with the base to facilitate deprotonation of compound (4) to form the reactive intermediate. Compound (5) may be obtained with a diastereomeric ratio of greater than 1:1, greater than 2:1, greater than 5:1, greater than 10:1, greater than 20:1, greater than 50:1 or greater than 99:1. The minor diastereomer may be removed via standard purification techniques such as, but not limited to, recrystallization and silica gel chromatography.

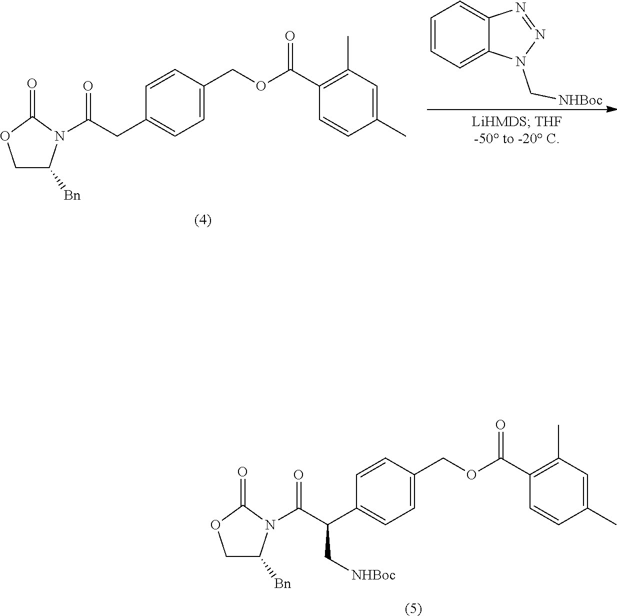 [SMILES: Cc1ccc(C(=O)OCc2ccc(CC(=O)N3C(=O)OC[C@H]3Cc3ccccc3)cc2)c(C)c1 ; CC(C)(C)OC(=O)NCn1nnc2ccccc21 ; "(4)" ; Cc1ccc(C(=O)OCc2ccc([C@@H](CNC(=O)OC(C)(C)C)C(=O)N3C(=O)OC[C@H]3Cc3ccccc3)cc2)c(C)c1 ; "(5)"]

Compound (5) can be converted to carboxylic acid (2) by addition of an appropriate nucleophilic base to remove the oxazolidinone chiral auxiliary. Suitably, the base is lithium hydroperoxide, which is formed in situ by reaction of lithium hydroxide with hydrogen peroxide. Use of lithium hydroperoxide is advantageous due to its effectiveness at removing the chiral auxiliary yet not hydrolyzing the benzyl ester.

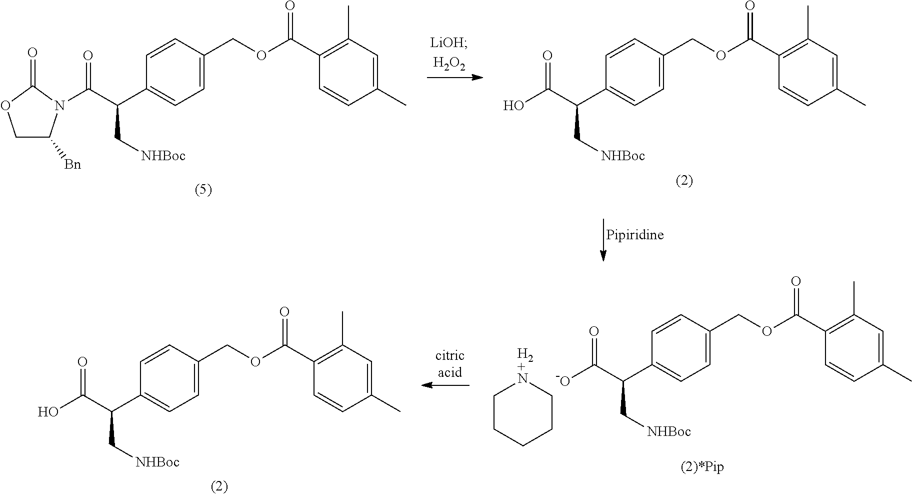

Compound (2), (S)-3-((tert-butoxycarbonyl)amino)-2-(4-(((2,4-dimethylbenzoyl)oxy)methyl)phenyl)propanoic acid, can be purified by converting it into a salt with a suitable base. Preferred bases are organic amines, such as but not limited to piperidine, and by crystallization of that salt in a suitable solvent or solvent system, such as, but not limited to, acetonitrile. The piperidine salt is known as the Compound (2) Pip salt, or piperidinium (S)-3-((tert-butoxycarbonyl)amino)-2-(4-(((2,4-dimethylbenzoyl)oxy)methyl)phenyl)propanoate. Other suitable amine bases for carrying out this transformation are: 2-aminoethanol, morpholine, piperidine, dibenzylamine, dicyclohexylamine, diisopropylamine, benzylamine, piperazine, DMAP. Other suitable inorganic bases are NaOH, LiOH·H₂O, Ca(OH)₂, Ba(OH)₂·8H₂O. Other suitable solvents and solvent systems include IPA, 1,4-dioxane, IPA+ heptanes, IPAc, and acetonitrile plus hepane. After purification, the Compound (2) Pip salt can be converted into the free acid form of compound (2) by, for example, dissolving the salt in ethyl acetate, MTBE or 2-MeTHF, then acidifying with citric acid to break the salt, followed by a crystallization of the free acid from heptane/ethyl acetate. Compound (2) can be then converted to compound (3) by activating the carboxylic acid group and reacting with 6-aminoisoquinoline. The carboxylic acid group may be activated by a variety of reagents and conditions, including conversion to a mixed anhydride or acid halide, or use of standard amide coupling reagents (e.g. EDCI, HOBT, DCC, DIC, HBTU, and HATU). Suitably, the carboxylic acid group is activated by formation of a mixed anhydride. The mixed anhydride can be formed by addition of an alkyl chloroformate such as trichlorodimethyl ethyl chloroformate and a base to form compound (3). In a preferred embodiment, trichlorodimethyl ethyl chloroformate and collidine are added to compound (2) at 0° C. in the presence of 6-aminoisoquinoline. A reactive mixed anhydride intermediate may form under such reaction conditions that may react with 6-aminoisoquinoline to form compound (3). The solvent employed may be DMF, alone or in combination with methylene chloride, or acetonitrile, and suitably, the solvent employed is DMF. Upon isolation, compound (3) can optionally be purified by silica gel column chromatography and/or recrystallization.

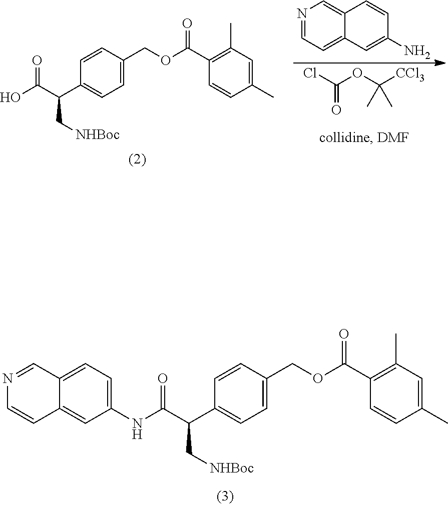

Conversion of compound (3) to compound (1) can be achieved by addition of a suitable reagent to remove the Boc protecting group. Suitably, an acid is used to remove the Boc protecting group. Any acid useful for removing the Boc protecting group may be used. The acid used for removing the Boc protecting group may also promote the formation of a salt of compound (1). The acid may be chosen so as to be advantageous for removal of the protecting group and also form a suitable pharmaceutically acceptable salt. Suitably, the acid employed in the conversion of compound (3) to compound (1) comprises at least two equivalents of methanesulfonic acid, resulting in the dimethanesulfonic acid salt of compound (1). Methanesulfonic acid is particularly useful because the desired product is formed in high yield with few byproducts and little decomposition. The dimethanesulfonic acid salt offers useful properties such as being easily purified, easy to handle and is able to be produced in large scale processes with great reproducibility.

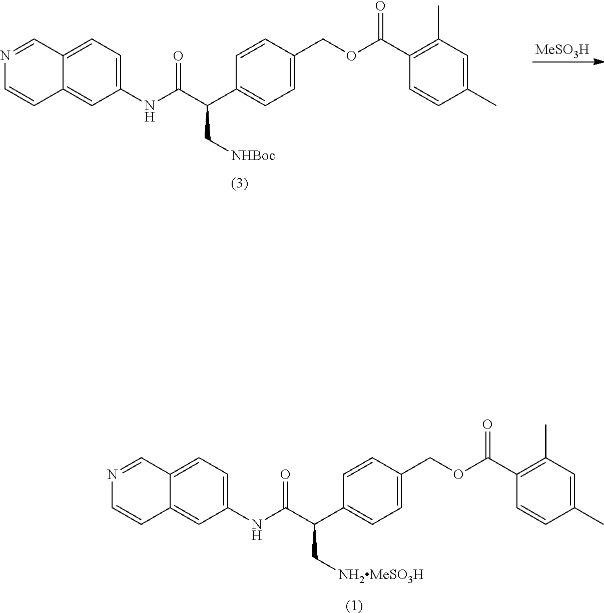

### Compounds of Formula (XI)

In another aspect, disclosed is a process for the synthesis of the compound of formula (XI):

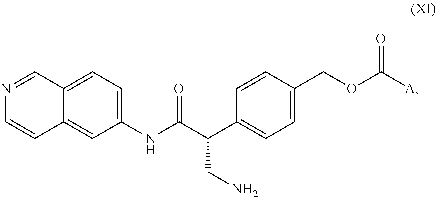

or a pharmaceutically acceptable salt; wherein A is cyclohexyl or phenyl, substituted with 0-3 substituents selected from the group consisting of alkyl, halogen, alkoxy, and cyano.

The process includes reacting 6-aminoisoquinoline with the compound of formula (XII), wherein PG is a protecting group for the nitrogen, to form the compound of formula (XIII). The compound of formula (XIII) can be transformed to the compound of formula (XI) by removal of the nitrogen protecting group. The nitrogen protecting group, PG, may be any suitable nitrogen protecting group known in the art. In certain embodiments, PG is selected from the group consisting of tert-butyloxycarbonyl (Boc), carbobenzyloxy (CBZ), 9-Fluorenylmethyl-oxycarbonyl (Fmoc).

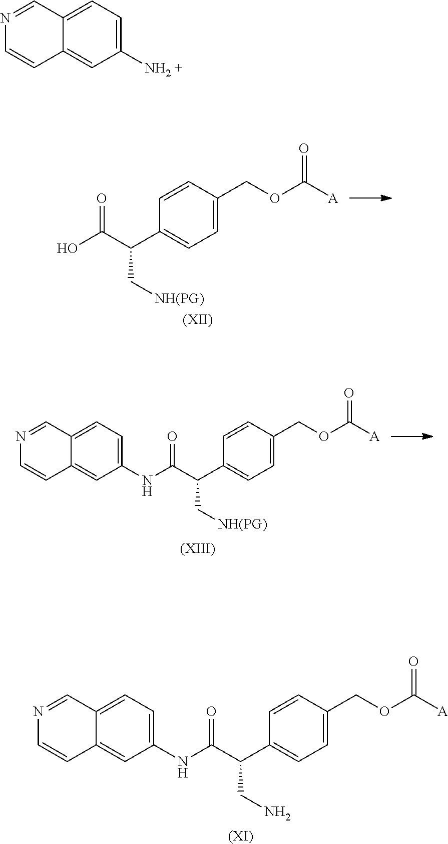

The process further includes the synthesis of the compound of Formula (XII). Aminoalkylation of the compound of Formula (IV), wherein T is a chiral auxiliary, can provide the compound of Formula (XV), which can be converted to the compound of Formula (XII) upon removal of the chiral auxiliary.

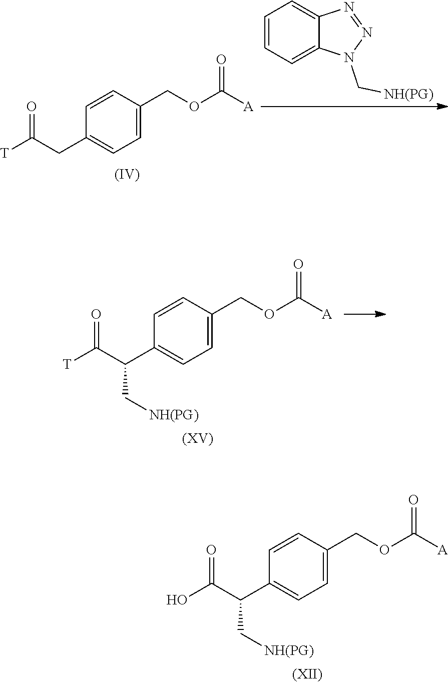

In certain embodiments, the compound of Formula (XI) may be obtained via the process described above for the synthesis of the compound of Formula (I). In particular, the compound of Formula (XV) may be formed in the conversion of the compound of Formula (IV) to the compound of Formula (V) as a minor product. Employing the reaction steps and schemes described above, the compound of Formula (XV) may, in turn, be transformed to the compound of Formula (XI). Accordingly, intermediate compounds, the compounds of Formulae (XII) and (XIII), may thus also be formed in the process.

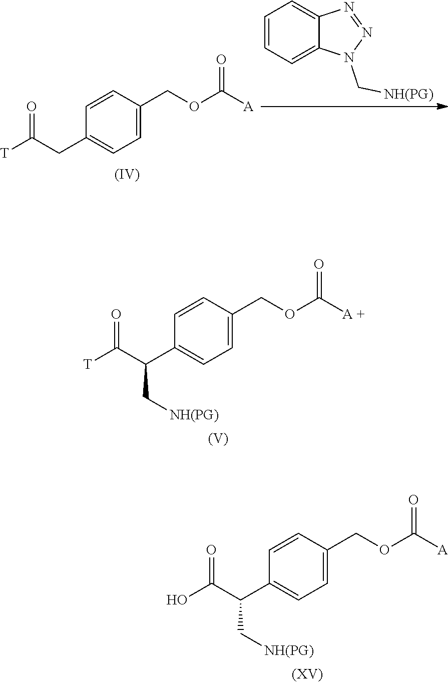

### Compounds of Formula (XI-a)

In an embodiment, the process for the synthesis of the compound of Formula (XI) is the process for the synthesis of the compound of Formula (XI-a):

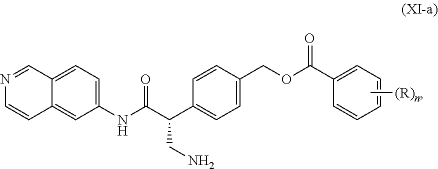

or a pharmaceutically acceptable salt; wherein each R is independently selected from the group consisting of C₁-C₄ alkyl, halogen, C₁-C₄ alkoxy and cyano; and n is an integer from 0 to 3. In one embodiment, the C₁-C₄ alkyl is a C₁-C₄ fluoroalkyl.

The process includes reacting 6-aminoisoquinoline with the compound of Formula (XII-a), wherein PG is a protecting group for the nitrogen, to form the compound of Formula (XIII-a). The compound of Formula (XIII-a) can be transformed to the compound of Formula (XI-a) by removal of the nitrogen protecting group. The nitrogen protecting group, PG, may be any suitable nitrogen protecting group known in the art. In certain embodiments, PG is selected from the group consisting of tert-butyloxycarbonyl (Boc), carbobenzyloxy (CBZ), 9-Fluorenylmethyl-oxycarbonyl (Fmoc).

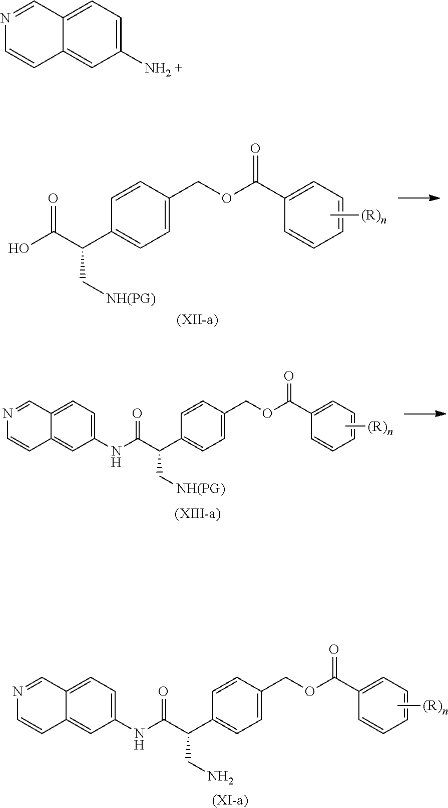

The process further includes the synthesis of the compound of formula (XII-a). Aminoalkylation of the compound of formula (IV-a), wherein T is a chiral auxiliary, can provide the compound of formula (XV-a), which can be converted to the compound of formula (XII-a) upon removal of the chiral auxiliary.

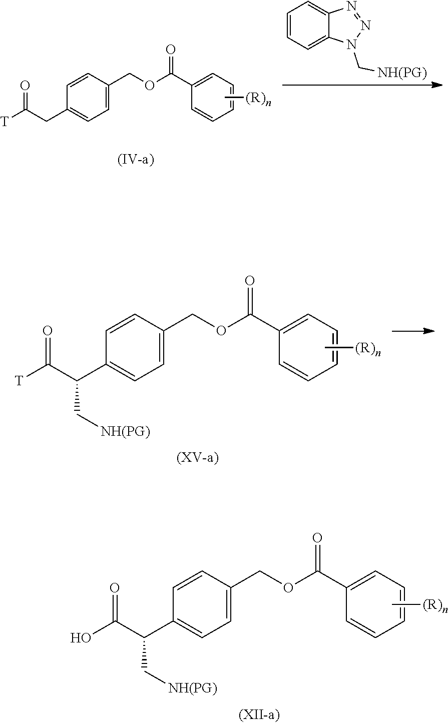

In certain embodiments, T may be the compound of formula (IX) o,

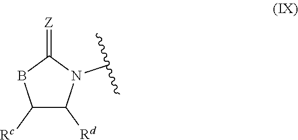

wherein Z is S or O; B is S or O; R^{c} is hydrogen, cycloalkyl, C₃-C₇ branched alkyl or aryl; R^{d} is C₁-C₄ alkyl, C₃-C₇ branched alkyl; arylalkyl or aryl; and R^{c} is C₁-C₄ alkyl or aryl.

In certain embodiments, T may be the compound of formula (IX-b),

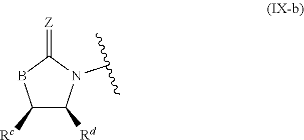

wherein Z is S or O; B is S or O; R^{c} is hydrogen or aryl; R^{d} is C₁-C₄ alkyl, arylalkyl or aryl; and R^{c} is C₁-C₄ alkyl or aryl.

In certain embodiments, T may be selected from the group consisting of

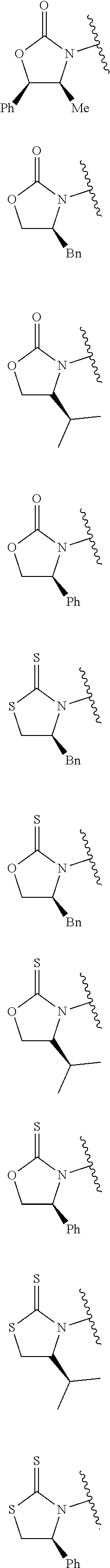

In a specific embodiment, T is

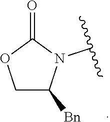 [SMILES: *N1C(=O)OC[C@@H]1Cc1ccccc1]

In certain embodiments, the compound of Formula (XI-a) may be obtained via the process described above for the synthesis of the compound of Formula (I-a). In particular, the compound of Formula (XV-a) may be formed in the conversion of the compound of Formula (IV-a) to the compound of Formula (V-a) as a minor product. Employing the reaction steps and schemes described above, the compound of Formula (XV-a) may, in turn, be transformed to the compound of Formula (XI-a). Accordingly, intermediate compounds, the compounds of Formulae (XII-a) and (XIII-a), may thus also be formed in the process.

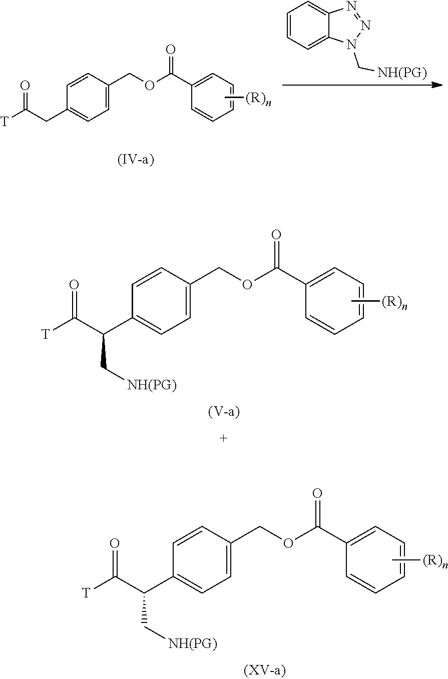

### Compound (11)

In an embodiment, the disclosed process for the synthesis of the compound of Formula (XI) is the process for the synthesis of compound (11):

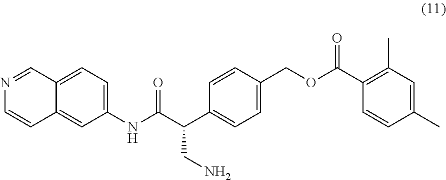 [SMILES: Cc1ccc(C(=O)OCc2ccc([C@H](CN)C(=O)Nc3ccc4cnccc4c3)cc2)c(C)c1]

or a pharmaceutically acceptable salt.

The process includes reacting 6-aminoisoquinoline with compound (12) to form compound (13). Compound (13) can be transformed to compound (11) by removal of the Boc protecting group.

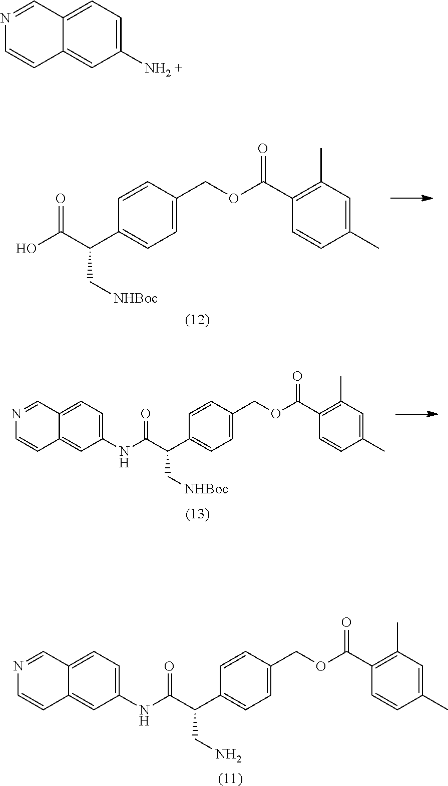

The process further includes the synthesis of compound (12). Addition of the chiral auxiliary to compound (8) can afford compound (14). Aminoalkylation of compound (14) can provide compound (15), which can be converted to compound (12) upon removal of the chiral auxiliary.

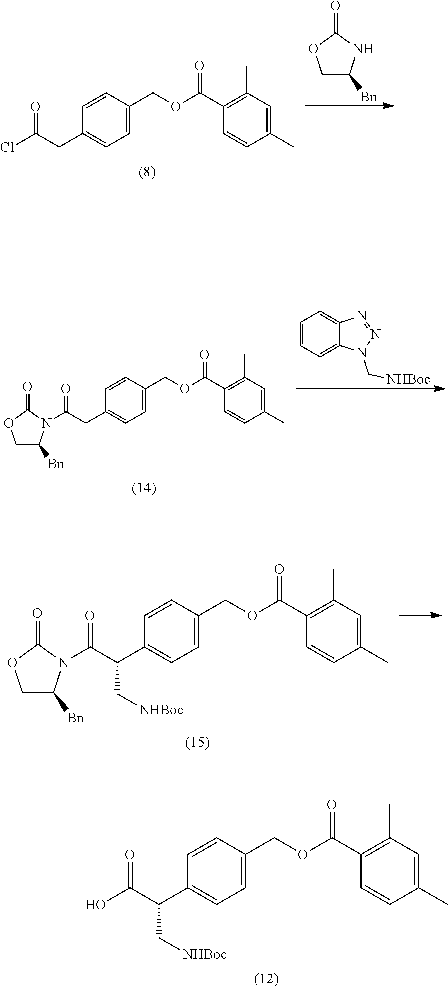 [SMILES: Cc1ccc(C(=O)OCc2ccc(CC(=O)Cl)cc2)c(C)c1 ; O=C1N[C@@H](Cc2ccccc2)CO1 ; Cc1ccc(C(=O)OCc2ccc(CC(=O)N3C(=O)OC[C@@H]3Cc3ccccc3)cc2)c(C)c1 ; CC(C)(C)OC(=O)NCn1nnc2ccccc21 ; Cc1ccc(C(=O)OCc2ccc([C@H](CNC(=O)OC(C)(C)C)C(=O)N3C(=O)OC[C@@H]3Cc3ccccc3)cc2)c(C)c1 ; Cc1ccc(C(=O)OCc2ccc([C@H](CNC(=O)OC(C)(C)C)C(=O)O)cc2)c(C)c1]

In certain embodiments, compound (11) may be obtained via the process described above for the synthesis of compound (1). In particular, compound (16) may be formed in the conversion of compound (4) to compound (5) as a minor product. Employing the reaction steps and schemes described above, compound (15) may, in turn, be transformed to compound (11). Accordingly, intermediate compounds, compounds (12) and (13), may thus also be formed in the process.

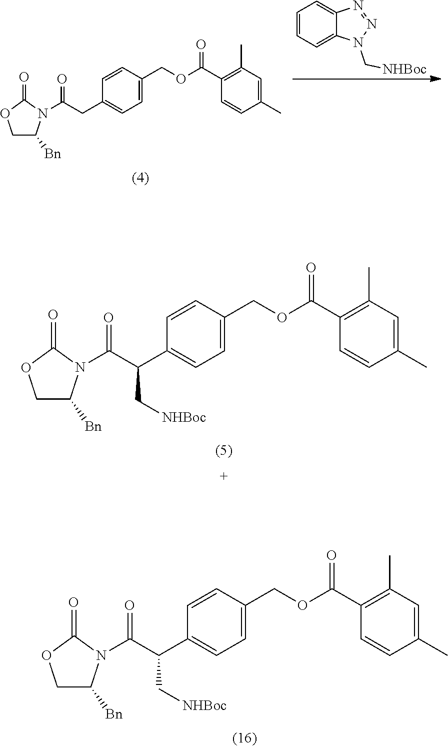 [SMILES: Cc1ccc(C(=O)OCc2ccc(CC(=O)N3C(=O)OC[C@H]3Cc3ccccc3)cc2)c(C)c1 ; CC(C)(C)OC(=O)NCn1nnc2ccccc21 ; Cc1ccc(C(=O)OCc2ccc([C@@H](CNC(=O)OC(C)(C)C)C(=O)N3C(=O)OC[C@H]3Cc3ccccc3)cc2)c(C)c1 ; Cc1ccc(C(=O)OCc2ccc([C@H](CNC(=O)OC(C)(C)C)C(=O)N3C(=O)OC[C@H]3Cc3ccccc3)cc2)c(C)c1]

In another aspect, the present disclosure provides a method for formation of an amide or ester bond comprising reacting an amine or alcohol with a carboxylic acid in the presence of

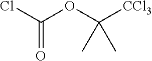

and a base. The amine and ester may be generally thought to be unreactive. In one embodiment, the amine is an aromatic amine. In one embodiment, the alcohol is an aromatic alcohol. The 1,1-dimethyl,2,2,2-trichloroethyl chloroformate may allow for stereoselective coupling of easily racemized carboxylic acids, particularly alpha-aromatic acids.

Abbreviations which have been used in the descriptions of the above structures and schemes include: Bn for benzyl; Ph for phenyl; Me for methyl; EDC for N-(3-Dimethylaminopropyl)-N′-ethylcarbodiimide hydrochloride; Boc for tert-butyl carbonyl; EDCI for 1-ethyl-3-(3-dimethylaminopropyl)carbodiimide, HOBT for hydroxybenzotriazole, CDI for carbonyl diimidazole; DCC for N,N′-dicyclohexylcarbodiimide; DIC for N,N′-disopropylcarbodiimide, HBTU for 2-(1H-benzotriazol-1-yl)-1,1,3,3-tetramethyluronium hexafluorophosphate; HATU for 1-[bis(dimethylamino)methylene]-1H-1,2,3-triazolo[4,5-b]pyridinium 3-oxid hexafluorophosphate; DMAP for dimethylaminopyridine; LiHMDS for lithium hexamethyldisilazide; NaHMDS for sodium hexamethyldisilazide; KHMDS for potassium hexamethyldisilazide; LDA for lithium diisopropylamide; DMF for dimethylformamide; and THF for tetrahydrofuran.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A compound of the instant disclosure may have at least one basic nitrogen whereby the compound can be treated with an acid to form a desired salt. For example, a compound may be reacted with an acid at low temperature to above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, hydrochloric, citric, or glutamic acid, and the like.

Optimum reaction conditions and reaction times for each individual step can vary depending on the particular reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using routine laboratory techniques and methods well known in the chemical literature. Routine experimentations, including appropriate manipulation of the reaction conditions, reagents and sequence of the synthetic route, protection of any chemical functionality that is incompatible with the reaction conditions, and deprotection at a suitable point in the reaction sequence of the method are included in the scope of the present disclosure. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4th ed.), John Wiley & Sons, NY (2006), which is incorporated herein by reference in its entirety. It can be appreciated that the synthetic schemes and specific examples as described are illustrative and are not to be read as limiting the scope of the present disclosure as it is defined in the appended claims. All alternatives, modifications, and equivalents of the synthetic methods and specific examples are included within the scope of the claims.

### Compounds

### Compounds of Formula (I)

In another aspect, disclosed herein are compounds of formula (I):

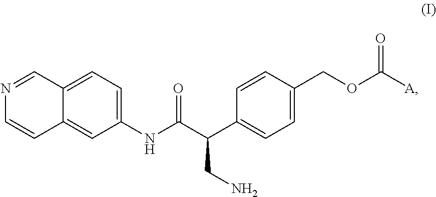

or a pharmaceutically acceptable salt; wherein A is cyclohexyl or phenyl, substituted with 0-3 substituents selected from the group consisting of alkyl, halogen, alkoxy, and cyano.

In an embodiment, the compound of Formula (I) is the compound of Formula (I-a):

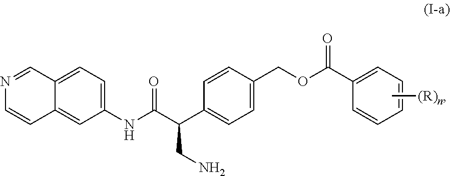

or a pharmaceutically acceptable salt; wherein each R is independently selected from the group consisting of C₁-C₄ alkyl, halogen, C₁-C₄ alkoxy and cyano; and n is an integer from 0 to 3.

In an embodiment, the compound of Formula (I) is compound (1):

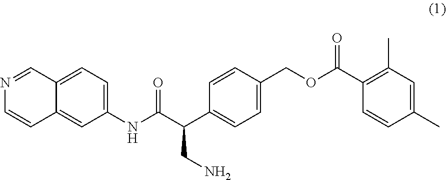 [SMILES: Cc1ccc(C(=O)OCc2ccc([C@@H](CN)C(=O)Nc3ccc4cnccc4c3)cc2)c(C)c1]

or a pharmaceutically acceptable salt.

In another aspect, disclosed herein are compounds of Formula (XI):

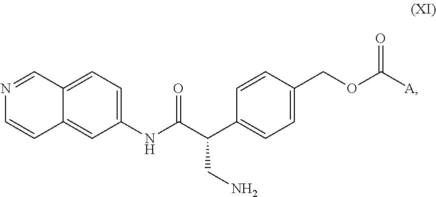

or a pharmaceutically acceptable salt; wherein A is cyclohexyl or phenyl, substituted with 0-3 substituents selected from the group consisting of alkyl, halogen, alkoxy, and cyano.

In an embodiment, the compound of Formula (XI) is the compound of Formula (XI-a):

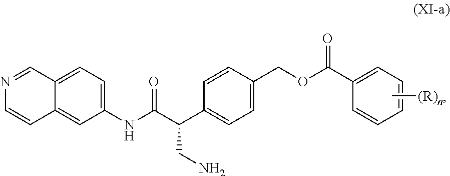

or a pharmaceutically acceptable salt; wherein each R is independently selected from the group consisting of C₁-C₄ alkyl, halogen, C₁-C₄ alkoxy and cyano; and n is an integer from 0 to 3.

In an embodiment, the compound of Formula (XI) is compound (11):

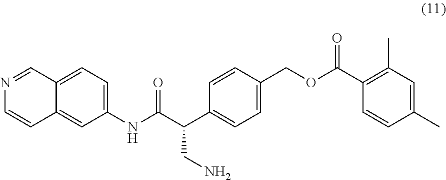 [SMILES: Cc1ccc(C(=O)OCc2ccc([C@H](CN)C(=O)Nc3ccc4cnccc4c3)cc2)c(C)c1]

or a pharmaceutically acceptable salt.

Compound names are assigned by using the structure naming algorithm as part of CHEMDRAW®.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this disclosure. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the present disclosure.

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the present disclosure are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

The compounds and processes of the of the instant disclosure will be better appreciated by reference to the following non-limiting examples, which are intended as an illustration of and not a limitation upon the scope of the present disclosure.

### Examples

Unless otherwise stated, temperatures are given in degrees Celsius (° C.); synthetic operations were carried out at ambient temperature, “rt,” or “RT,” (typically a range of from about 18-25° C.); evaporation of solvents was carried out using a rotary evaporator under reduced pressure (typically, 4.5-30 mm Hg) with a bath temperature of up to 60° C.; the course of reactions was typically followed using thin layer chromatography (TLC); all melting points, if given, are uncorrected; all intermediates as well as the final product exhibited satisfactory ¹H-NMR, HPLC and/or microanalytical data; and the following conventional abbreviations are used: L (liter(s)), mL (milliliters), mmol (millimoles), g (grams), mg (milligrams), min (minutes), and h (hours).

Proton magnetic resonance (¹H NMR) spectra were recorded on either a Varian INOVA 600 MHz (¹H) NMR spectrometer, Varian INOVA 500 MHz (¹H) NMR spectrometer, Varian Mercury 300 MHz (¹H) NMR spectrometer, or a Varian Mercury 200 MHz (¹H) NMR spectrometer. All spectra were determined in the solvents indicated. Although chemical shifts are reported in ppm downfield of tetramethylsilane, they are referenced to the residual proton peak of the respective solvent peak for ¹H NMR. Interproton coupling constants are reported in Hertz (Hz).

### Example 1: Preparation of 2-(4-(((2,4-Dimethylbenzoyl)oxy)methyl)phenyl)acetic acid (9)

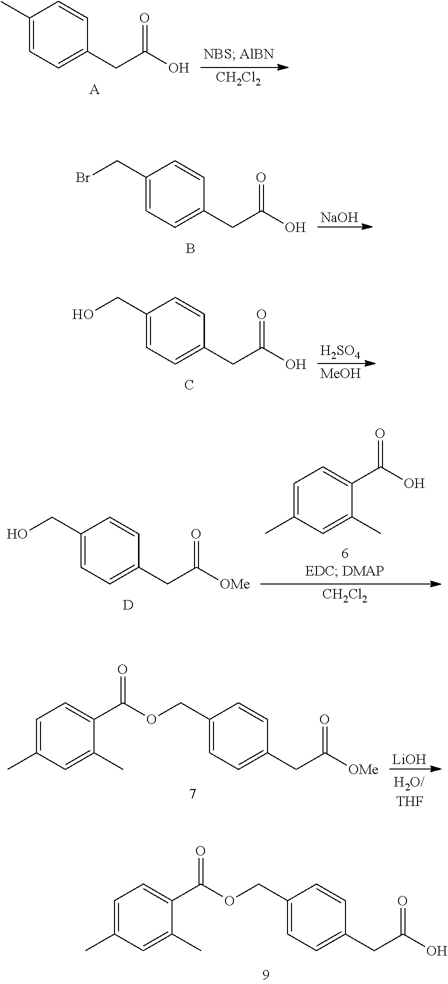

2-(4-(Bromomethyl)phenyl)acetic acid (B): To a solution of A (4.4 kg, 29.3 mol, 1 eq) in acetonitrile (22 L) was added N-bromosuccinimide (NBS) (5740 g, 32.2 mol, 1.1 eq) and azobisisobutyronitrile (AIBN) (9.2 g, 0.02 eq). The resulting mixture was slowly heated to 80° C. and stirred for 15-30 min. After the starting 1 was consumed as indicated by TLC, the reaction mixture was cooled to −5° C. slowly and kept at −5° C. overnight. The resulting solid was collected by filtration. The filter cake was washed with petroleum ether/EtOAc (1:1) (5 L), petroleum ether (5 L×2), saturated NaHSO₃ (aq.) (5 L), water (5 L), and petroleum ether (5 L) to give the title compound (2.3 kg, yield: 34.2%). HPLC purity: 96.8% (254 nm); ¹H NMR (300 MHz, DMSO-d6) δ 12.3 (s, 1H), 7.4 (d, J=8.0 Hz, 2H), 7.2 (d, J=8.0 Hz, 2H), 4.7 (s, 2H), 3.57 (s, 2H).

2-(4-(Hydroxymethyl)phenyl)acetic acid (C): To a solution of NaOH (1.61 kg, 40.2 mol, 4 eq) in water (90 L) was added B (2.3 kg, 10.0 mol, 1 eq) and the resulting mixture was stirred at RT overnight. TLC analysis indicated consumption of B. The reaction mixture was then carefully acidified with concentrated H₂SO₄ (1.0 L) to pH˜2. Then, solid NaCl (25 kg) was added to the mixture followed by extraction with EtOAc (33 L×3). The combined organic phase was washed with brine, dried over Na₂SO₄, and concentrated until a significant amount of solid precipitated. The resulting suspension was kept at ˜4-6° C. overnight to allow for further crystallization. The solid product was then collected by filtration. The filter cake was washed with petroleum ether (2 L×2) to yield the title compound (1.2 kg, yield: 71.9%). HPLC purity: 97.8% (220 nm); ¹H NMR (300 MHz, DMSO-d6) δ 12.27 (s, 1H), 7.26-7.12 (m, 4H), 5.14 (s, 1H), 4.47 (s, 2H), 3.53 (s, 2H).

Methyl 2-(4-(hydroxymethyl)phenyl)acetate (D): To a solution of C (2.5 kg, 15.06 mol, 1 eq) in MeOH (15 L) was slowly added concentrated H₂SO₄ (1.5 L) at 0° C. The resulting mixture was allowed to stir at RT overnight. After C was consumed as indicated by TLC, the reaction mixture was poured into water (20 L) and extracted with EtOAc (20 L×3). The combined organic layers were washed with saturated NaHCO₃ solution (aq.) (20 L×3) and then brine (20 L). The organic phase was dried over Na₂SO₄, filtered and concentrated to give the title compound (2.2 kg) as a viscous oil. HPLC purity: 90% (220 nm); ¹H NMR (300 MHz, CDCl₃) δ 7.35-7.28 (m, 4H), 4.68 (s, 2H), 3.70 (s, 3H), 3.64 (s, 2H).

4-(2-Methoxy-2-oxoethyl)benzyl 2,4-dimethylbenzoate (7): A solution of 2,4-dimethylbenzoic acid (6) (2.01 kg, 13.4 mol, 1.1 eq) and EDC (4.2 kg, 21.9 mol, 1.8 eq) in dichloromethane was stirred at RT for 1 h. D (2.2 kg, 12.2 mol, 1 eq) and 4-dimethylaminopyridine (DMAP) (298 g, 2.44 mol, 0.2 eq) were added to the reaction mixture, which was allowed to stir at RT overnight. After consumption of D was complete as judged by TLC, the reaction mixture was washed three times with 1 N HCl solution (16 L×3), then once with brine (16 L). The separated organic layer was dried over Na₂SO₄, filtered, and concentrated. The crude product was recrystallized in MeOH to afford the title compound (2.32 kg, yield 60.9%). HPLC purity: 98.6% (210 nm); ¹H NMR (300 MHz, CDCl3) δ 7.88 (d, J=7.8 Hz, 1H), 7.42 (d, J=8.0 Hz, 2H), 7.31 (d, J=8.0 Hz, 2H), 7.05 (m, 2H), 5.32 (s, 2H), 3.72 (s, 3H), 3.64 (s, 2H), 2.60 (s, 3H), 2.36 (s, 3H).

2-(4-(((2,4-Dimethylbenzoyl)oxy)methyl)phenyl)acetic acid (9): To a solution of 7 (1.2 kg, 3.85 mol, 1 eq) in THF (2.4 L) was added a solution of LiOH (176 g, 4.2 mol, 1.1 eq) in water (3.6 L) dropwise over 1 h. The resulting mixture was allowed to stir for 1.5 h. TLC analysis indicated the consumption of 7. The reaction mixture was washed with MTBE (2.5 L×4). The aqueous layer was acidified with a saturated citric acid aqueous solution (550 mL) to pH ˜3-4, which forms a precipitate. The resulting admixture was concentrated by rotary evaporator to remove the organic solvents. The solid product was then collected by filtration. The crude product was slurried in water (3.5 L) for 30 min. After filtration, the collected solid was then slurried in heptane (5 L) to produce the title compound (2.05 kg, yield: 89.6%). HPLC purity: 100% (210 nm); LCMS (ESI−): m/z=297 (M-1). ¹H NMR (300 MHz, CDCl3) δ 7.88 (d, J=7.8 Hz, 1H), 7.43 (d, J=8.0 Hz, 2H), 7.32 (d, J=8.0 Hz, 2H), 7.05 (m, 2H), 5.32 (s, 2H), 3.69 (s, 2H), 2.59 (s, 3H), 2.36 (s, 3H). The compound may be recrystallized from water/acetone if the analytical data indicate the presence of citric acid.

### Example 2: Preparation of DBMPA Imide

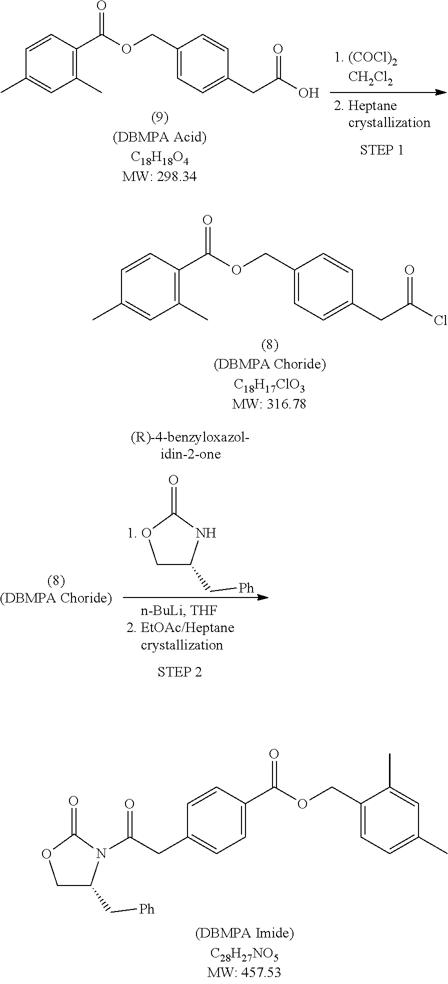

Step 1: Preparation of DBMPA Chloride, 4-(2-chloro-2-oxoethyl)benzyl 2,4-dimethylbenzoate (8). Starting material DBMPA Acid, (9) (5.0 kg assay-corrected, 1 equiv.) is dissolved in 5.5 volumes of dichloromethane and converted to DBMPA chloride (8) by treatment with oxalyl chloride (1.15 equiv.). The reaction is stirred at 20±5° C. for 16 to 24 hours. Additional oxalyl chloride may be added (up to 0.2 equiv.) to ensure reaction completion (IPC analysis by HPLC or TLC). The dichloromethane is exchanged with heptane and the total process volume is adjusted to about ˜11 volumes with heptane. The resulting suspension of DBMPA chloride (8) is cooled to 0±5° C. DBMPA chloride (8) is isolated as a crystalline solid, washed with additional heptane (2×2.5 volumes) and dried under nitrogen for NLT 2 hours. The material is dissolved in 2 volumes of tetrahydrofuran (THF) and taken directly into the next reaction.

Step 2: Preparation of DBMPA Imide, (7). The subsequent equivalents for Step 2 are based on the isolated DBMPA Chloride. In a separate vessel, the chiral auxiliary, Compound A, 0.95 equiv. is dissolved in THF (7.5 volumes) and cooled to −80±5° C. n-Butyl lithium in heptane (1.05 equiv.) is then added at a rate such that the internal temperature does not exceed −65° C. The mixture is stirred at −70±5° C. for NLT 15 minutes. The DBMPA chloride solution is added to the anion of the chiral auxiliary (along with a 0.5 volume THF rinse) maintaining an internal temperature of −70±5° C. The reaction mixture is stirred at −70±5° C. for NLT 15 to NMT 60 minutes until the reaction is complete (IPC by HPLC, was TLC). The reaction mixture is then quenched with 2 volumes of 10% aqueous ammonium chloride and warmed to 15-30° C. for NLT 30 minutes, but NMT 20 hours. The bottom aqueous layer is removed, and the organic layer is concentrated by distillation under reduced pressure to about 2 process volumes. The remaining THF is exchanged with ethyl acetate. Following purified water and 14% aqueous sodium chloride washes, the organic layer is concentrated by distillation and adjusted to about 6 volumes with ethyl acetate. Heptane (10 volumes) is added and the product is crystallized from ethyl acetate/heptane with seeding. The suspension is cooled to 5±5° C. and the product is isolated by filtration, washed with heptane (2×5 vol.) and dried to constant weight in a pre-heated vacuum oven at 40±5° C. for NLT 10 hours to provide DBMPA Imide (bulk weight change NMT 1%) as a white to off-white solid. The solid is sampled for in-process testing (IPC by HPLC) and may be taken into Step 3 or re-crystallized from ethyl acetate/heptane as described below, based on the in-process test results. There is no IPC after a second crystallization. This intermediate may also be stored based on supporting hold-time data.

An alternate Step 2 procedure utilizes sodium hydride (NaH) in place of n-butyllithium. In this process, the chiral auxiliary is dissolved in THF and added to a suspension of NaH (60% in mineral oil, 1.1 eq assay corrected) in THF at 20±5° C. The deprotonation/anion formation is allowed to progress at 20±5° C. for 15-30 minutes and then at 20-25° C. for up to 35 hours. The resulting mixture is cooled to 3±5° C. followed by addition of a solution of DBMPA Chloride dissolved in THF over 30-60 minutes. The resulting reaction mixture is allowed to stir at 3±5° C. for 2-4 hours before analyzing (IPC by HPLC or TLC) and progressing into a workup as described above.

Re-crystallization: Volumes below are based on the isolated imide, but reflect similar concentrations described above for the initial isolation from ethyl acetate/heptane. DBMPA Imide is dissolved in 3.6 volumes of ethyl acetate. The solution is then adjusted to 20±5° C. Heptane (7.3 volumes) is added, and the product is crystallized from ethyl acetate/heptane with seeding. The suspension is cooled to 5±5° C. and the product is isolated by filtration, washed with heptane (2×3.6 vol.) and dried to constant weight in a pre-heated vacuum oven at 40±5° C. for NLT 10 hours to provide DBMPA Imide as a white to off-white solid.

### Example 3: Preparation of N-Boc DBMPP Acid

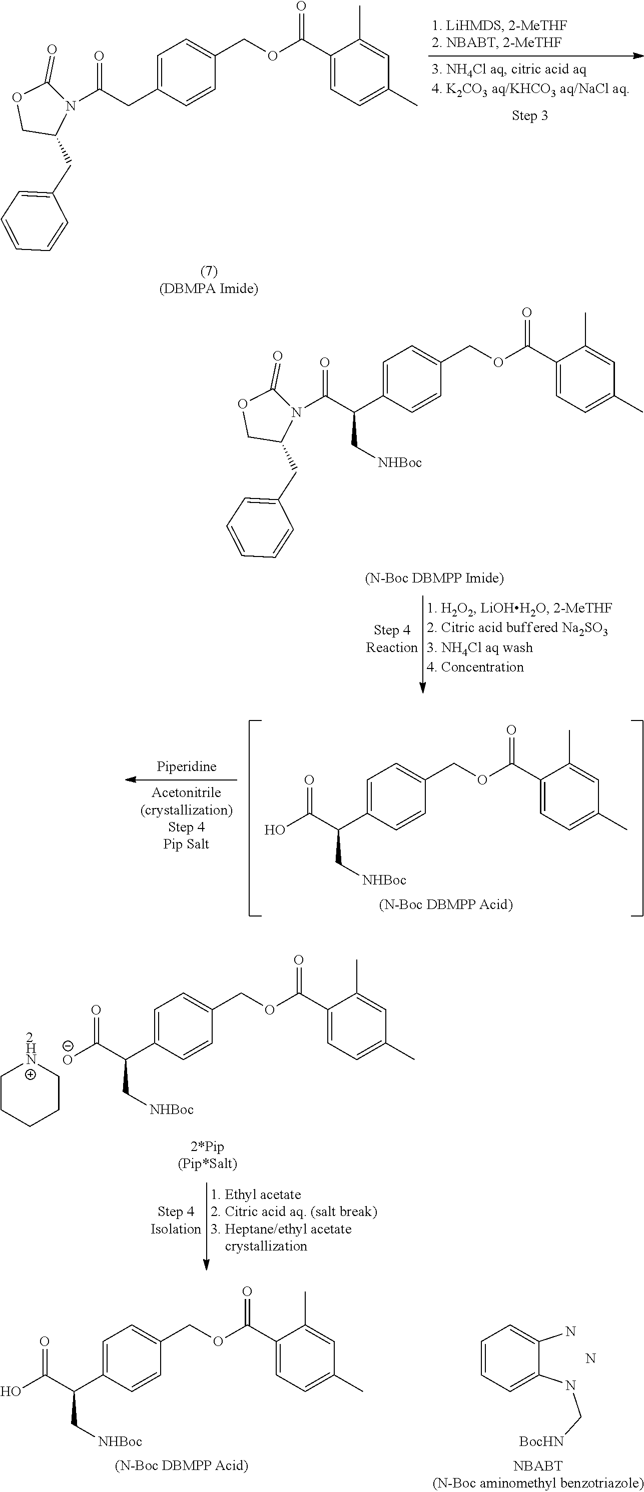

Step 3: Preparation of N-Boc DBMPP Imide, crude. Charge DBMPA imide (1 equiv) followed by anhydrous 2-methyl THF (2-Me THF, 10 volumes NMT 100 ppm water) in the reactor. The solution is cooled to −25±5° C. Add LiHMDS 1.0 M solution in THF (1.2 equiv) to the DBMPA imide solution maintaining batch temperature below −15° C. Stir the reaction mixture for 30-90 min at −25±5° C. Separately, charge N-Boc aminomethylbenzotriazole (NBABT, 1.2 equiv) in another reactor and add anhydrous 2-MeTHF (6 volumes). The water content of the NBABT solution by KF should be <100 ppm (IPC). If KF is >100 ppm, distillative-dry the solution using anhydrous 2-Me THF. Transfer NBABT solution to the reactor maintaining temperature below −15° C. Stir the reaction mixture for 1.5 to 3 hrs. at −25±5° C. Monitor the progress of the reaction by HPLC.

After completion of the reaction, the mixture is quenched at 25±5° C. with 10% aqueous ammonium chloride (2 volumes) followed by 10% aqueous citric acid (2 vol). Adjust the reaction mixture temperature to 10-20° C. and age 1-16 hrs. Allow layers to separate NLT 15 min and split layers leaving rag layer with organic phase. Wash organic layer with 10% aqueous NaCl (2 volumes) and stir biphasic mixture for NLT 15 min. Allow layers to separate NLT 15 min and split layers leaving rag layer with organic phase. Wash organic layer with 10% aqueous K₂CO₃ (3×10 volumes) followed by 10% aqueous KHCO₃ (1×10 volumes) and 10% aqueous NaCl (1×10 volumes). Stir biphasic mixture NLT 15 min and allow layers to separate for NLT 15 min and split layers leaving rag layer with organic phase. Concentrate organic phase to ˜10 volumes. This will be N-Boc DBMPP Imide crude solution.

Step 4: Preparation of N-Boc DBMPP Acid. Charge the crude solution from Step 3 in the reactor and add water (5 volumes). Cool the mixture to 0±5° C. Once cooled, charge hydrogen peroxide (4 equiv) in one portion. Charge in the reactor lithium hydroxide monohydrate solution (1.2 equiv in 1.3 volumes of water) in one portion and stir the reaction at 0±5° C. for NLT 6 hrs. Monitor the progress of the reaction (IPC by HPLC, was TLC).

Quench the reaction with 10% sodium sulfite solution (20 volumes) buffered with citric acid, target pH 6-7. Adjust the temperature to 25±10° C. and stir NLT 16 hrs. Stop stirring and allow layers to separate NLT 15 min and split layers leaving rag layer with organic phase. Wash organic phase with 10% aq. ammonium chloride (3×13 volumes). Concentrate the organic phase under vacuum with vessel jacket temperature NMT 40° C. to ˜5 volumes. Charge acetonitrile (2×8 volumes) and concentrate to ˜5 volumes. Charge additional acetonitrile to bring total of ˜10 volumes. Cool the mixture to 20±5° C. and age for NLT 30 min. Pass the solution through clarifying filter and collect in a clean vessel.

Charge piperidine (0.41 equiv) at 20±5° C. in the solution over NLT 10 min. Seed the solution with 0.20 wt % of chirally pure seed in acetonitrile (0.5×volumes), stir the solution at 20±5° C. and age for 2-4 hrs. Charge remainder piperidine (0.59 eq) at 20±5° C. for 4-6 hrs., and age the mixture 12-16 hrs. at 20±5° C. Filter the content and wash the cake with acetonitrile (2 volumes), mix for NLT 10 min and filter. Dry the cake at NMT 50° C. for 6 hrs. to get piperidine salt (IPC—chiral and achiral; rec-crystallization is performed if needed based on IPC). No additional IPC testing if re-crystallization is performed.

Step 4: Salt Break and Free Acid Crystallization. For the calculations in this step, the calculation ratio herein is based on the Compound 2 Pip*salt. Slurry the piperidine salt in ethyl acetate (7 volumes) and charge to vessel. Wash the mixture with 10% aqueous citric acid (2×10 volumes). Allow layers to separate NLT 15 min and split layers discarding the lower aqueous layer. Wash the organic phase with water (2×10 volumes). Allow layers to separate NLT 15 min and split layers discarding the lower aqueous layer. Cool the organic phase to 0±5° C. and add heptane (7 volumes) over 15 min. Seed the solution with 1 wt % N-Boc DBMPP acid seed slurried in 9:1 v/v heptane:ethyl acetate (0.5×volumes). Then, add additional heptane (14 volumes) over 6-8 hrs. Age the solution for NMT than 1 hr after heptane addition completion. Filter the product and wash the cake with 9:1 v/v heptane:ethyl acetate (4 volumes). Dry the product under vacuum at 50±5° C. until dry (IPC chiral and achiral; re-crystallization is performed if needed based on IPC). No additional IPC testing if re-crystallization is performed. Yield: 60-80% cumulative (65-80% Step 4)

### Example 4: Netarsudil Synthesis—Preparation of N-Boc Netarsudil (Compound 7)

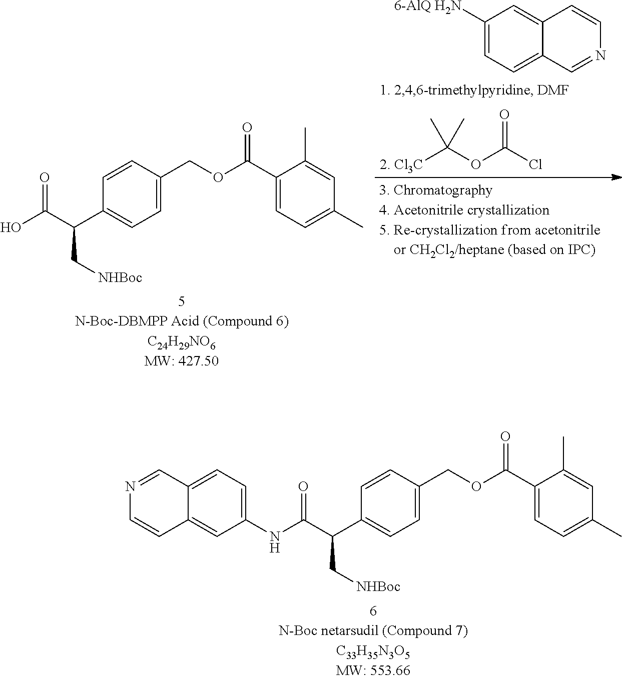

Step 5: Preparation of N-Boc Netarsudil Compound (7). N-Boc DBMPP Acid (approximately 2.0 kg, 1 equiv.) and 6-AIQ (1.3 equiv.) are dissolved in N,N-Dimethylformamide (DMF, 13 volumes). 2,4,6-Trimethylpyridine (collidine, 1.3 equiv.) is added and the reaction mixture is cooled to 0±5° C. A solution of 2,2,2-trichloro-1,1-dimethylethyl chloroformate (1.3 equiv.) in 3 volumes of DMF is added to the reaction mixture as rapidly as possible followed by a 1 volume DMF rinse. The reaction is stirred at 0±5° C. for 6-8 hours.

The reaction is then quenched with 20 volumes of a 10% aqueous KHCO₃ solution. Ethyl acetate (30 volumes) is charged to the reactor and the temperature is adjusted to 20±5° C. for NLT 10 minutes.

The aqueous layer is removed, and the organic layer washed with 10 volumes of aqueous 10% citric acid solution. The aqueous layer is again removed, and the organic layer is washed with 10 volumes of 10% aqueous KHCO₃ solution. The temperature of the biphasic mixture is adjusted to 40±5° C. for NLT 10 minutes. The aqueous layer is removed, and the remaining organic layer is stirred at 40±5° C. for NLT 10 hours and NMT 20 hours. The reaction mixture is then cooled to 20±5° C. and any remaining aqueous layer is removed. The mixture is then concentrated to about 4 volumes through vacuum distillation and further reduced to an oil via rotary evaporation. The residue is dissolved in 2 volumes of dichloromethane for purification by silica gel chromatography, for example a Biotage 400 L System, using 40 kg Columns packed with HP Sphere silica, with a silica gel to starting material ratio of NLT 12.5:1 (silica gel: starting material).

Impurities are eluted with 40:60 ethyl acetate:heptane and then the product is eluted with 70:30 ethyl acetate:heptane. The major fractions containing the Compound 7 as determined by HPLC, was TLC) are combined, concentrated under reduced pressure to about 10 volumes and then the solvent is exchanged with acetonitrile to about 33 volumes. The internal temperature is adjusted to 70±5° C. and the solids are dissolved. The solution is then cooled to 55±2° C., seeded with a sample of N-Boc Netarsudil (1-5 wt %) and cooled further to 20±2° C. Filtration of the product commences NMT 1 hour after reaching 20±2° C. The cake is washed with acetonitrile (2×2 volumes) and then dried at 50±5° C. in vacuo to constant weight to provide Compound 7 as a white to light yellow solid. The product is sampled for in-process achiral and chiral purity. (IPC chiral and achiral; re-crystallization, if needed, based on IPC)

If the criteria for chiral purity are not met, the product is re-crystallized from 30 volumes of acetonitrile (with respect to isolated Compound 7)

If the criteria for achiral chromatographic purity are not met (independent of chiral purity), the product is re-crystallized from dichloromethane/heptane as described below.

The product is dissolved in 10 volumes of dichloromethane at 20±5° C. Heptane (4 volumes) is added over NLT 15 minutes while maintaining a temperature of 20±5° C. The solution is seeded with 1-5 wt % Compound 7 and stirred at 20±5° C. for NMT 1 hour. Heptane (11 volumes) is then added over 70-120 minutes. The resulting mixture is stirred at 20±5° C. for NMT 4 hours after seed addition. The crystallized product is then isolated by filtration. The cake is washed with heptane (2×2 volumes) and dried at 50±5° C. in vacuo to constant weight to provide N-Boc Netarsudil as a white to light yellow solid. This intermediate may be taken directly into Step 6 or stored based on supporting hold-time data. Yield: 40-80% Average Yield on 250-L Scale: 68%.

### Example 5: Netarsudil Synthesis—Preparation of Netarsudil as its Dimesylate Salt

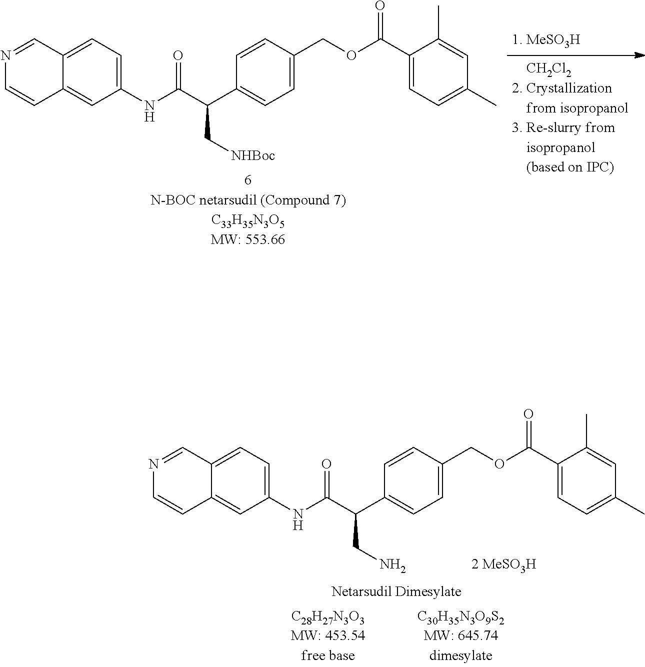

Step 6: Preparation of Netarsudil-Reaction and Initial Isolation. Compound 7 (approximately 1.4 kg, 1 equiv., assay-corrected) is dissolved in 15 volumes of dichloromethane and passed through a clarifying filter. The filter is washed with 2×2 volumes of dichloromethane, and the combined filtrates are adjusted to a temperature of 20±5° C. Methanesulfonic acid (2.5 equiv.) is dissolved in 2.5 volumes of dichloromethane and added through a filter into the reactor at such a rate that the reaction temperature is maintained at 20±5° C. A 2.5 volume line rinse is similarly added. The reaction mixture is then stirred at 20±5° C. for NLT 48 hours. The temperature is adjusted to 40±5° C. and the mixture is stirred at this temperature for 2-4 hours until the reaction is complete (IPC, HPLC was TLC). The reaction mixture is concentrated to about 5 volumes via vacuum distillation. The remaining dichloromethane is exchanged with isopropanol via successive distillations to 25 volumes. The reaction mixture is warmed to 70±5° C. The solution is cooled to 50±2° C. at a rate of about −0.3° C./min. Netarsudil dimesylate seed (0.5-5.5 wt %) is suspended in 30 volumes of isopropanol. The suspended seed is then added to the reactor and the mixture is stirred at 50±2° C. for NLT 1 hour. The mixture is cooled to 20±5° C. at a rate of about −0.3° C./minute then stirred at this temperature for NLT 1 hour. The resulting slurry is warmed to 50±5° C. and stirred at this temperature for NLT 90 minutes. The mixture is re-cooled to 20±5° C. at a rate of about −0.3° C./minute and stirred for 1-24 hours. The product is collected by filtration and crystalline solids are washed with isopropanol (3 volumes).

The solids are dried on the filter for 3-16 hours with nitrogen flow through the filter cake. The solids are transferred to a pre-heated oven set to 30±5° C. for 16-24 hours. The oven temperature is slowly increased to 69±10° C. and drying continues at this temperature for 24 to 48 hours. The solids are de-lumped using a knife mill, sampled for purity (IPC achiral by HPLC), then returned to the oven at 69±10° C. for 60-132 hours (or for NLT 48 hours if the in-process testing for HPLC purity fails). The material is then tested for residual isopropanol and water content (IPC by GC and KF).

An agitated filter dryer may be used as a replacement for the knife mill and tray dryer in this process. An agitated filter dryer typically requires up to 72 hrs total time.

The material may optionally be re-slurried in isopropanol based on the in-process testing for purity or isopropanol content.

Re-slurry. Netarsudil is suspended in 25 volumes of filtered isopropanol and the temperature is adjusted to 50±5° C. The mixture is stirred at this temperature for about 2 hours and then cooled to 20±5° C. at a rate of about −0.15° C./minute. The cooled suspension is stirred at 20±5° C. for 8 to 24 hrs. The product is collected by filtration and washed with filtered isopropanol (3 volumes).

The solids are dried on the filter for 3-16 hours with a dry nitrogen flow through the filter cake. The solids are transferred to a pre-heated oven set to 30±5° C. for 16-24 hours. The oven temperature is slowly increased to 69±10° C. and drying continues at this temperature for 24 to 48 hours. The solids are de-lumped using a knife mill, sampled for purity (by HPLC) and returned to the oven at 69±10° C. for an additional 72±12 hour. The light yellow to white powder is then dried until the residual isopropanol level is NMT 6000 ppm by in-process testing. An agitated filter dryer may be used to replace the knife mill and tray dryer treatment of this product.

The final drug substance is hygroscopic and is packaged at a temperature of <25° C. with relative humidity 40%. Yield: 80-99%. Average Yield on 250-L Scale: 93%.

In conclusion, to meet the ongoing need for commercial-scale supplies of the glaucoma drug netarsudil, a process has been developed that offers improved chemical yields and, importantly, scaleability.

It is understood that the foregoing detailed description and accompanying examples are merely illustrative and are not to be taken as limitations upon the scope of the present disclosure.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the present disclosure, may be made without departing from the spirit and scope thereof.

What is claimed is:

1. A method of synthesizing a compound of Formula (I-a): 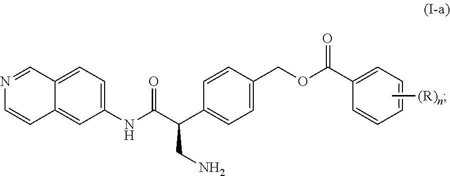
wherein each R is independently selected from the group consisting of C₁-C₄alkyl, halogen, C₁-C₄ alkoxy and cyano; and n is an integer from 0 to 3;
comprising:
(a) reacting a compound of Formula (II-a), wherein PG is a nitrogen protecting group, 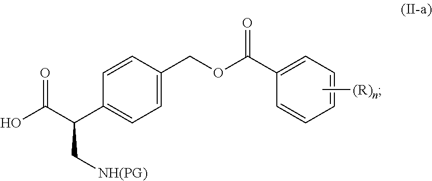
with 6-aminoisoquinoline to form a compound of Formula (III-a) 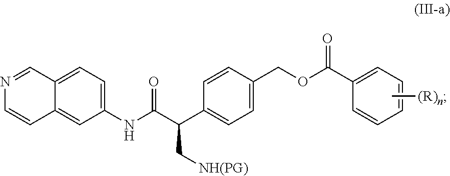
wherein each R is independently selected from the group consisting of C₁₋₄ alkyl, halogen, C₁₋₄ alkoxy and cyano; and n is an integer from 0 to 3; and
(b) removing the nitrogen protecting group to form the compound of Formula (I-a).

2. The method of claim 1, further comprising:
(a) reacting a compound of Formula (IV-a), 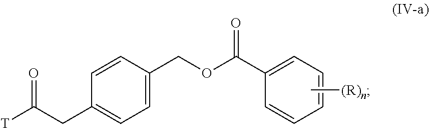
wherein each R is independently selected from the group consisting of C₁₋₄ alkyl, halogen, C₁₋₄ alkoxy and cyano; n is an integer from 0 to 3; and T is a chiral auxiliary; with 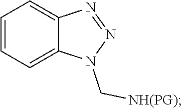
wherein PG is a nitrogen protecting group;
to form a compound of Formula (V-a): 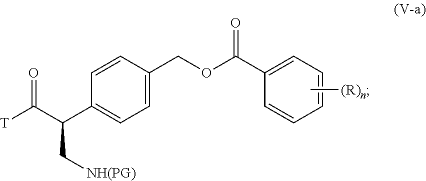
wherein each R is independently selected from the group consisting of C₁₋₄ alkyl, halogen, C₁₋₄ alkoxy and cyano; n is an integer from 0 to 3; and T is a chiral auxiliary; and
(b) removing the chiral auxiliary to form the compound of Formula (II-a).

3. The method of claim 2, further comprising converting a compound of Formula (VIII-a): 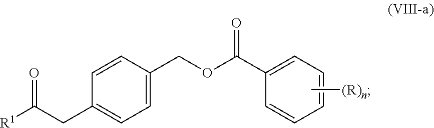
wherein each R is independently selected from the group consisting of C₁-C₄alkyl, halogen, C₁-C₄ alkoxy and cyano; n is an integer from 0 to 3; and R₁ is halogen, OR^{a}, OC(O)R^{b}, SR^{a}, or SC(O)R^{b}; wherein R^{a} is H, alkyl or aryl, and R^{b} is alkyl or aryl;
to the compound of Formula (IV-a): 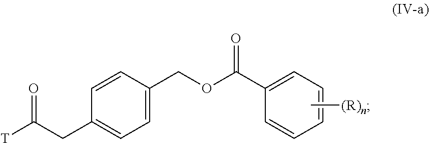
wherein each R is independently selected from the group consisting of C₁-C₄alkyl, halogen, C₁-C₄ alkoxy and cyano; n is an integer from 0 to 3; and T is a chiral auxiliary.

4. An amine salt of (S)-3-((tert-butoxycarbonyl)amino)-2-(4-(((2,4-dimethylbenzoyl)oxy)methyl)phenyl)propanoic acid, chosen from the list of 2-aminoethanol, morpholine, piperidine, dibenzylamine, dicyclohexylamine, diisopropylamine, benzylamine, piperazine, DMAP.

5. The method of any one of claims 1-3, wherein n is 2 and each R is methyl.

6. The method of any one of claims 1-3, wherein the compound of Formula (I-a) is

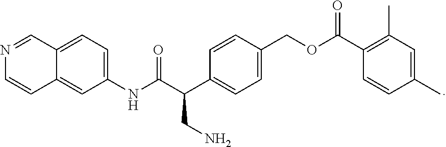 [SMILES: Cc1ccc(C(=O)OCc2ccc([C@@H](CN)C(=O)Nc3ccc4cnccc4c3)cc2)c(C)c1]

7. The method of any one of claims 5-6, wherein the compound of Formula (VIII-a) is

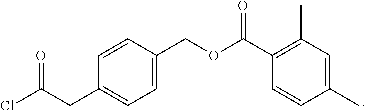 [SMILES: Cc1ccc(C(=O)OCc2ccc(CC(=O)Cl)cc2)c(C)c1]

8. The method of any one of claims 1-3, wherein n is 0.

9. The method of anyone of claims 5-8, wherein T is 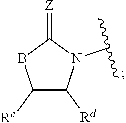
wherein
Z is S or O;
B is S or O;
R^{c} is hydrogen, cycloalkyl, C₃-C₇ branched alkyl or aryl;
R^{d} is C₁-C₄ alkyl, C₃-C₇ branched alkyl; arylalkyl or aryl.

10. The method of claim 9, wherein T is 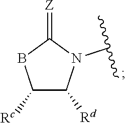
wherein
Z is S or O;
B is S or O;
R^{c} is hydrogen or aryl;
R^{d} is C₁-C₄ alkyl, arylalkyl or aryl.

11. A method of purifying (S)-3-((tert-butoxycarbonyl)amino)-2-(4-(((2,4-dimethylbenzoyl)oxy)methyl)phenyl)propanoic acid, wherein (S)-3-((tert-butoxycarbonyl)amino)-2-(4-(((2,4-dimethylbenzoyl)oxy)methyl)phenyl)propanoic acid is reacted to form an amine salt, and the salt is recrystallized and then the purified salt is then converted back into (S)-3-((tert-butoxycarbonyl)amino)-2-(4-(((2,4-dimethylbenzoyl)oxy)methyl)phenyl)propanoic acid.

12. The method of claim 11, wherein the amine used to form the amine salt is piperidine.

13. A method of synthesizing a compound of the following formula 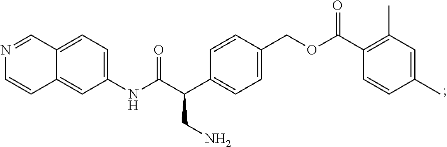 [SMILES: Cc1ccc(C(=O)OCc2ccc([C@@H](CN)C(=O)Nc3ccc4cnccc4c3)cc2)c(C)c1]
comprising:
(a) reacting 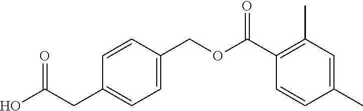 [SMILES: Cc1ccc(C(=O)OCc2ccc(CC(=O)O)cc2)c(C)c1]
with a chlorinating agent, to form an acid chloride;
(b) crystallizing the acid chloride from n-heptane to form a purified acid chloride;
(c) reacting the purified acid chloride with 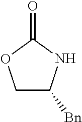 [SMILES: O=C1N[C@H](Cc2ccccc2)CO1]
in the presence of a base, to form 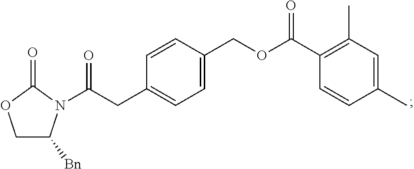 [SMILES: Cc1ccc(C(=O)OCc2ccc(CC(=O)N3C(=O)OC[C@H]3Cc3ccccc3)cc2)c(C)c1]
(d) reacting the product of step (c) with 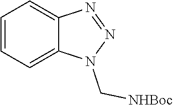 [SMILES: CC(C)(C)OC(=O)NCn1nnc2ccccc21]
in the presence of a base at a temperature between −50° C. and −0° C. to form 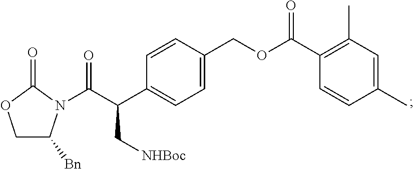 [SMILES: Cc1ccc(C(=O)OCc2ccc([C@@H](CNC(=O)OC(C)(C)C)C(=O)N3C(=O)OC[C@H]3Cc3ccccc3)cc2)c(C)c1]
(e) reacting the product of step (d) with LiOOH to form 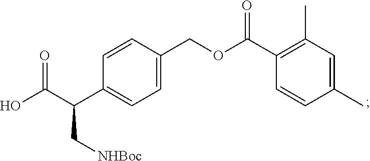 [SMILES: Cc1ccc(C(=O)OCc2ccc([C@@H](CNC(=O)OC(C)(C)C)C(=O)O)cc2)c(C)c1]
(f) formation of an amine salt and then recrystallizing the product of step (e) to form a purified product of step (e);
(g) activating the carboxylic acid group of the purified product of step (f) and reacting the activated carboxylic acid with 6-aminoisoquinoline to form 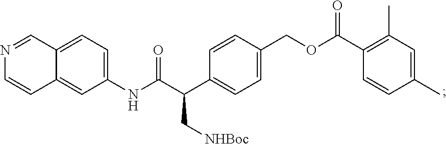 [SMILES: Cc1ccc(C(=O)OCc2ccc([C@@H](CNC(=O)OC(C)(C)C)C(=O)Nc3ccc4cnccc4c3)cc2)c(C)c1]
and
(h) recyrstallizing the product of step (g);
(i) reacting the product of step (h) with at least 2 equivalents of MeSO₃H to form

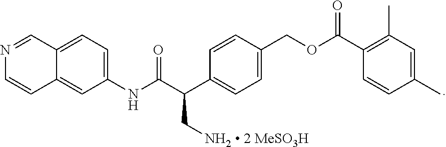

14. The method of claim 13, wherein the solvent used for the purification of the amine salt of step (e) is acetonitrile.

15. The method of claim 13, wherein the base of step (c) is NaH, LiH, KH, nBuLi, secBuLi, LiHMDS, NaHMDS, or KHMDS and step (b) is performed at a temperature between −90° C. and −50° C.

16. The method of claim 13, wherein the base of step (d) is NaH, LiHMDS or NaHMDS.

17. The method of claim 13, wherein the LiOOH is formed in situ by lithium hydroxide and hydrogen peroxide.

18. The method of claim 13, wherein the carboxylic acid group is activated in step (g) by addition of trichlorodimethyl ethyl chloroformate and collidine at a temperature of about 0° C.

19. The method of claim 18, wherein the purified product of step (f), 6-aminoisoquinoline, and collidine are mixed followed by addition of trichlorodimethyl ethyl chloroformate.

20. The method of claim 18, wherein the carboxylic acid group is activated by conversion to a mixed anhydride intermediate.

21. A compound, selected from:

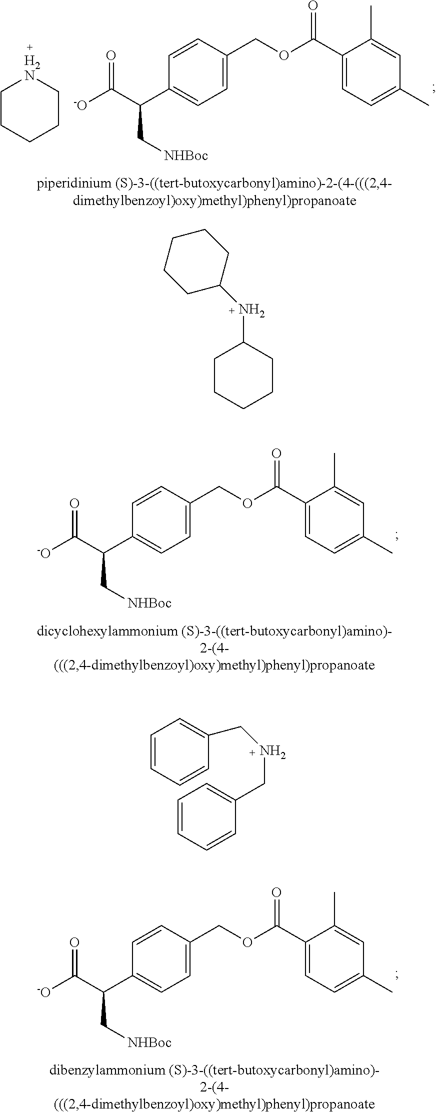

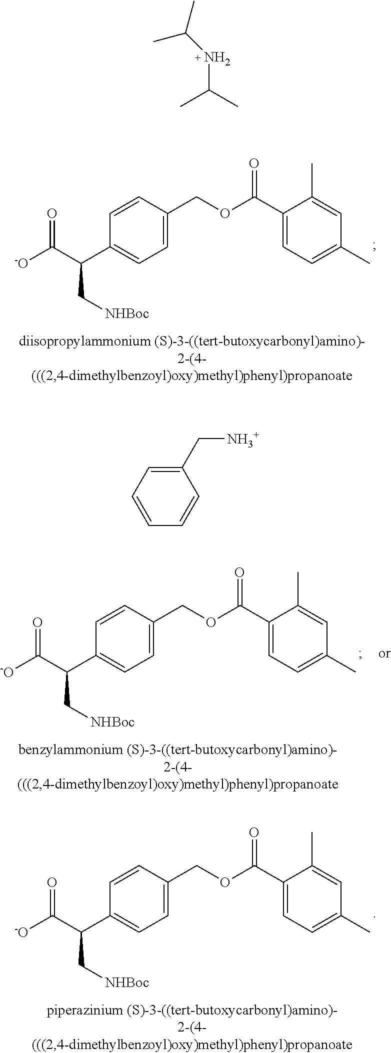

22. The compound of claim 21, which is a solid form of

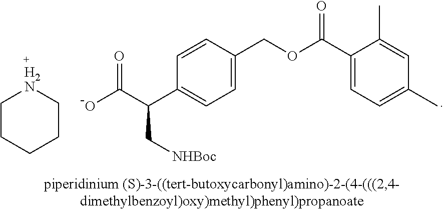

23. The compound of claim 22, wherein the solid form is a crystalline form.

24. The compound of claim 21, which is a solid form of the compound.

25. The compound of claim 25, wherein the solid form is a crystalline form.

26. A compound described herein, prepared by one or more processes described herein.

27. A composition, comprising the compound of one of claims 21-26.
